(19) United States
(12) Patent Application Publication
(10) Pub. No.: US 2015/0140649 A1
(43) Pub. Date: May 21, 2015

(54) NUCLEIC ACID EXTRACTION DEVICE

(71) Applicant: Seiko Epson Corporation, Tokyo (JP)

(72) Inventors: Masato HANAMURA, Shiojiri (JP); Kotaro IDEGAMI, Chino (JP); Yuji SAITO, Shiojiri (JP); Kiyohito YAMADA, Shiojiri (JP)

(21) Appl. No.: 14/547,621

(22) Filed: Nov. 19, 2014

(30) Foreign Application Priority Data
Nov. 20, 2013 (JP) 2013-239671

(51) Int. Cl. C12N 15/10

(52) U.S. Cl. CPC C12N 15/1013
USPC 435/293.1; 422/527

(57) ABSTRACT

A nucleic acid extraction device includes: a tube section in which are disposed in this order a first plug formed of oil, a second plug formed of a washing liquid immiscible with oil and for washing a substance adsorbing a nucleic acid, a third plug formed of oil, a fourth plug formed of an elution liquid immiscible with oil and for eluting the nucleic acid from the substance, and a fifth plug formed of oil; and a cover section disposed around the tube section.

### BACKGROUND

1. Technical Field

The present invention relates to a nucleic acid extraction device.

2. Related Art

Gene remedies such as genetic diagnosis and gene therapy have attracted interest with the recent development of gene technology. In this connection, a variety of gene techniques for variety determination and breeding have been developed in the field of agriculture and livestock farming. A wide range of techniques are available in gene technology, including PCR (Polymerase Chain Reaction). PCR has now become indispensable for understanding information about biological materials. PCR is a technology used to amplify the nucleic acid of interest by thermal cycling of a solution (reaction liquid) containing the nucleic acid to be amplified (target nucleic acid), and reagents. Typical PCR thermal cycling involves two or three temperature steps.

The diagnosis of infections such as influenza as currently practiced at clinics most commonly uses a simplified test kit such as immunochromatography. However, simplified test kits are not always satisfactory in terms of accuracy, and the use of the more accurate PCR for infection diagnosis is desired. In outpatient clinics or other such healthcare facilities, the limited consultation time limits the time that can be spent on testing. For example, an influenza test such as by simplified immunochromatography as currently practiced saves time at the expense of test accuracy.

Under these circumstances, the need for a shorter reaction time has risen in medical settings requiring a more accurate test by PCR. Apparatuses for reducing a PCR reaction time are available. For example, JP-A-2009-136250 discloses a biological sample reaction apparatus in which a biological sample reaction chip charged with a reaction liquid and a liquid immiscible with the reaction liquid and having a smaller specific gravity than the reaction liquid is rotated about a horizontal rotational axis to move the reaction liquid for thermal cycling. Various other PCR techniques are also available, including a technique using magnetic beads (JP-A-2009-207459), and a technique that uses magnetic beads as a means to move droplets, and that moves droplets in a temperature varying region of a substrate for PCR thermal cycling (JP-A-2008-012490).

Studies intended to reduce the PCR thermal cycle time are available as described above. However, the current techniques to reduce the PCR start-up time that includes the extraction of template nucleic acids from a specimen are not necessarily sufficient. For example, PCR requires extracting template nucleic acids (DNA: deoxyribonucleic acid, and/or RNA: ribonucleic acid) from a specimen (such as blood, sinus mucus, and oral mucosa) (hereinafter, such a procedure is also referred to simply as “pretreatment”). As such, simply reducing the PCR thermal cycle time is not sufficient, and the demand from the clinic cannot be fully met unless the time for extracting nucleic acids (pretreatment) is reduced.

The pretreatment typically uses a column and magnetic beads. However, the procedures including dispensing, stirring, and centrifuging reagents all require manual operations, or expensive large-scale equipment such as an auto-extraction device. In either case, the pretreatment is a laborious process, requiring at least 30 minutes. Taking the pretreatment time into consideration, the total test time from the collection of a specimen to the finding of the test result amounts to about 1 hour, at the shortest, even when the PCR thermal cycle alone is completed in a short time period (for example, within 15 minutes).

It has thus been practically impossible to perform all the procedures from the extraction of nucleic acids (pretreatment) to the PCR thermal cycle at the clinic, where the consultation time is limited. This is indeed one of the obstacles preventing the wide use of PCR testing across healthcare facilities. Specifically, the time required for the PCR itself and for the pretreatment, and the complexity of these processes have prevented, at least in part, the use of PCR in the clinic, despite that PCR has been known to offer more sensitive and accurate testing than immunochromatography.

### SUMMARY

An advantage according to some aspects of the invention is to provide a nucleic acid extraction device that reduces the time required for the PCR pretreatment.

A nucleic acid extraction device according to an aspect of the invention includes: a tube section in which are disposed in this order a first plug formed of oil, a second plug formed of a washing liquid immiscible with oil and for washing a substance adsorbing a nucleic acid, a third plug formed of oil, a fourth plug formed of an elution liquid immiscible with oil and for eluting the nucleic acid from the substance, and a fifth plug formed of oil; and a cover section disposed around the tube section.

The cover section may be detachable, and the tube section may be expandable and compressible in a direction of extension of the tube section. The tube section and the cover section may be separated from each other. The distance from an inner cavity surface of the tube section to an outer surface of the cover section is preferably 3 mm or more. The cover section may have a slit that extends along a direction of extension of the tube section. The cover section may have a hole. The cover section may be made of a deformable material, and a gas may be sealed between the tube section and the cover section to prevent the tube section and the cover section from adhering to each other. The cover section may contain a non-magnetic substance selected from metals and metal alloys.

A nucleic acid extraction device according to another aspect of the invention includes a tube section in which are disposed in this order a first plug formed of oil, a second plug formed of a washing liquid immiscible with oil and for washing a substance adsorbing a nucleic acid, a third plug formed of oil, a fourth plug formed of an elution liquid immiscible with oil and for eluting the nucleic acid from the substance, and a fifth plug formed of oil, the tube section having a side wall with a thickness of 3 mm or more. The side wall of the tube section may contain a non-magnetic substance selected from metals and metal alloys.

As used herein, “plug” as in “liquid plug” refers to a shape occupied substantially solely by a liquid along the longitudinal direction of a tube or a tube section, and indicates a state in which the “plug” compartmentalizes the inner space of a tube or a tube section. As used herein, “substantially” means that minute amounts of other substances (such as a liquid) may be present (for example, in the form of a thin film) around the plug, specifically on the inner wall of a tube or a tube section. The terms “tube” or “tube section” refer to a cylindrical portion, or a deformable tubular portion having an inner cavity of a cross sectional shape that allows a liquid to maintain the plug in the tube or tube section.

### BRIEF DESCRIPTION OF THE DRAWINGS

Embodiments of the invention will be described with reference to the accompanying drawings, wherein like numbers reference like elements.

FIG. 1 is a diagram schematically illustrating part of a nucleic acid extraction device according to an embodiment.

FIG. 2 is a diagram schematically illustrating part of a nucleic acid extraction device according to an embodiment.

FIG. 3 is a diagram schematically illustrating part of a nucleic acid extraction device according to an embodiment.

FIG. 4 is a diagram schematically illustrating a nucleic acid extraction device according to an embodiment.

FIG. 5 is a diagram schematically illustrating a nucleic acid extraction device according to an embodiment.

FIG. 6 is a diagram schematically illustrating a nucleic acid extraction device according to an embodiment.

FIG. 7 is a diagram schematically illustrating a nucleic acid extraction device according to an embodiment.

FIG. 8 is a diagram schematically illustrating a nucleic acid extraction device according to an embodiment.

FIG. 9A is a diagram schematically illustrating a nucleic acid extraction device according to an embodiment, and FIG. 9B is a cross sectional view taken at broken line of FIG. 9A.

FIG. 10A is a diagram schematically illustrating a nucleic acid extraction device according to an embodiment, and FIG. 10B is a cross sectional view taken at broken line of FIG. 10A.

FIG. 11A is a diagram schematically illustrating a nucleic acid extraction device according to an embodiment, and FIG. 11B is a cross sectional view taken at broken line of FIG. 11A.

FIG. 12 is a diagram schematically illustrating a nucleic acid extraction device according to an embodiment.

FIG. 13 is a diagram schematically illustrating part of a nucleic acid extraction device according to an embodiment.

FIG. 14 is a diagram schematically illustrating an example of a nucleic acid extraction kit according to an embodiment.

FIG. 15 is a diagram schematically illustrating an example of a nucleic acid extraction kit according to an embodiment.

FIG. 16 is a schematic diagram explaining a variation of a nucleic acid extraction method of an embodiment.

FIG. 17 is a perspective view illustrating an example of a nucleic acid extraction apparatus according to an embodiment.

FIG. 18 is a perspective view illustrating an example of a nucleic acid extraction apparatus according to an embodiment.

FIG. 19 is a graph showing results of PCR in an example 1 according to an embodiment.

FIG. 20 is a graph showing the relationship between elution temperature and DNA yield.

### DESCRIPTION OF EXEMPLARY EMBODIMENTS

Some embodiments of the invention are described below. The following embodiments are solely intended to illustrate the invention. The invention is in no way limited by the following exemplary embodiments, and includes various modifications as may be made within the gist of the invention. It should also be noted that the configurations described below do not all necessarily represent essential constituting elements of the invention.

### 1. NUCLEIC ACID EXTRACTION DEVICE

The nucleic acid extraction device 1000 of the present embodiment includes a tube section 100, and a first plug 10, a second plug 20, a third plug 30, a fourth plug 40, and a fifth plug 50.

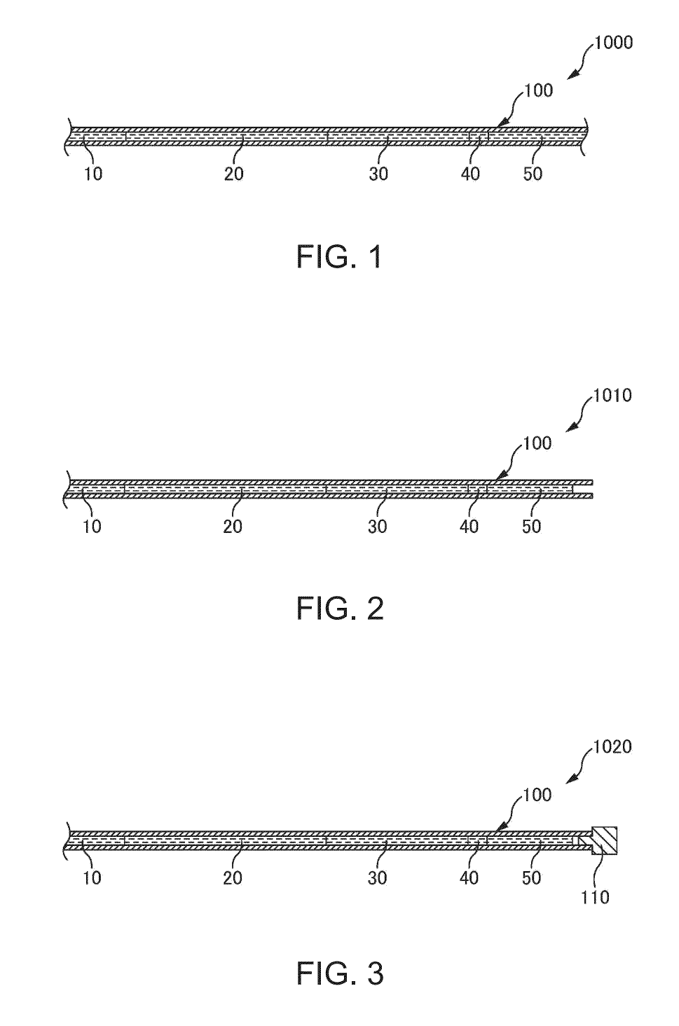

FIG. 1 is a diagram schematically representing part of the nucleic acid extraction device 1000 of the present embodiment.

### 1.1. Tube Section

A tube section 100 represents part of the nucleic acid extraction device 1000. The nucleic acid extraction device 1000 may be configured to include other members, in addition to the tube section 100. For example, the nucleic acid extraction device 1000 may include members such as a pipe, a container, a stopper, a joint, a pump, and a control unit connected to the tube section 100.

The tube section 100 is a cylindrical section with an inner cavity that allows for passage of a liquid along a longitudinal direction. The tube section 100 has a longitudinal direction, but may be bent. The size and shape of the inner cavity of the tube section 100 are not particularly limited, as long as the liquid therein can maintain the shape of a plug in the tube section 100. The size of the inner cavity, and the cross sectional shape perpendicular to the longitudinal direction of the tube section 100 may vary along the longitudinal direction of the tube section 100. Whether the liquid can maintain the shape of a plug inside the tube section 100 depends on conditions such as the material of the tube section 100, and the type of the liquid, and as such the cross sectional shape perpendicular to the longitudinal direction of the tube section 100 is designed in a way that allows the liquid to maintain the shape of a plug in the tube section 100.

The outer cross sectional shape perpendicular to the longitudinal direction of the tube section 100 is not particularly limited. The thickness of the tube section 100 (the length from the side surface of the inner cavity to the outer surface) is not particularly limited either. When the cross section perpendicular to the longitudinal direction of the inner cavity of the tube section 100 is circular, the inner diameter (the diameter of a circle in a cross section perpendicular to the longitudinal direction of the inner cavity) of the tube section 100 may be, for example, 0.5 mm to 3 mm. This inner diameter range of the tube section 100 is preferable because it makes it easier for the liquid to form a plug in a wide range of tube section 100 materials and liquid types.

The material of the tube section 100 is not particularly limited, and may be, for example, glass, polymer, or metal. It is, however, preferable that a glass or polymer material that is transparent to visible light is selected as the material of the tube section 100, because such materials allow visual access to the inside (cavity) of the tube section 100 from outside. It is also preferable to select a magnetically transparent material or a non-magnetic material as the material of the tube section 100 because such materials allow magnetic particles to pass through the tube section 100 under externally applied magnetic force when passing magnetic particles.

The first to fifth plugs 10 to 50 are disposed inside the tube section 100, in this order. The first plug 10 is formed of oil, the second plug 20 is formed of a first washing liquid immiscible with oil, the third plug 30 is formed of an oil immiscible with the first washing liquid, the fourth plug 40 is formed of an elution liquid immiscible with oil, and the fifth plug 50 is formed of an oil immiscible with the elution liquid.

### 1.2. First Plug, Third Plug, and Fifth Plug

The first plug 10, the third plug 30, and the fifth plug 50 are all formed of oil. The first plug 10, the third plug 30, and the fifth plug 50 may be of different oils. The liquids of the first plug 10, the second plug 20, the third plug 30, the fourth plug 40, and the fifth plug 50 are selected so that the liquids are immiscible with each other between the adjacent plugs.

The oil may be, for example, silicone oil or mineral oil. As used herein, “silicone” means an oligomer or polymer with a siloxane bond backbone. In this specification, the term “silicone oil” is used to refer to particularly silicones that are in a liquid state in a temperature range used for the thermal cycle process. In this specification, the term “mineral oil” is used to refer to oils purified from petroleum, and that are liquid in a temperature range used for the thermal cycle process. These oils are preferable for elevated PCR because these have high heat stability, and are available as products with a viscosity of, for example, 5×10³ Nsm⁻² or less.

Examples of the silicone oil include dimethyl silicone oils such as KF-96L-0.65cs, KF-96L-1cs, KF-96L-2cs, and KF-96L-5cs available from Shin-Etsu Silicone, SH200 C FLUID 5 CS available from Dow Corning Toray Co., Ltd., and TSF451-5A, and TSF451-10 available from Momentive Performance Materials Inc., Japan. The mineral oil is, for example, an oil containing alkane of about 14 to 20 carbon atoms as the primary component. Specific examples include n-tetradecane, n-pentadecane, n-hexadecane, n-heptadecane, n-octadecane, n-nonadecane, and n-tetracosane.

As described above, it is preferable to add an antistatic agent to the oil. The antistatic agent may be, for example, a modified silicone oil. Here, “modified silicone oil” means a silicone oil having a substituent. The antistatic agent preferably has, for example, a carbinol group, an alkylsilyl group, a fluoroalkyl group, a silanol group, or an alkylsilsesquioxy group as a substituent. The antistatic agent may have more than one of these substituents, for example, an alkylsilyl group and an alkylsilsesquioxy group, or an alkylsilyl group and a fluoroalkyl group. It is also possible to use cyclic siloxane. More preferably, the antistatic agent has a heat stable property in the temperature range of the thermal cycle process. Examples include carbinol-modified silicone oil, KF-6001 (Shin-Etsu Silicone), BY 16-201, 5562 CALBINOL FLUID (Dow Corning Toray Co., Ltd.), and XF42-B0970 (Momentive Performance Materials Inc., Japan). The carbinol-modified silicone oil has a viscosity of 3×10⁴ Nsm⁻² or more, a little high for elevated PCR when used alone. However, because the volume resistance value is lower than that of dimethyl silicone oil, the conductivity of the oil can be adjusted by mixing dimethyl silicone oil. Specifically, the specific electrical resistance decreases as more carbinol-modified silicone oil is added. The amount of carbinol-modified silicone oil is not particularly limited; however, the oil as a mixture preferably has a specific electrical resistance value of 5.4×10¹⁰ Ω·cm or less.

The antistatic agent may be a liquid containing more than one component, or a mixture of more than one liquid. For example, the antistatic agent may be X21-5250 (trimethylsiloxysilicate 50%, cyclopentasiloxane 50%), or X21-5616 (trimethylsiloxysilicate 60%, isododecane 40%) available from Shin-Etsu Silicone.

The second plug 20 is disposed between the first plug 10 and the third plug 30. Another liquid plug may be disposed on the side the first plug 10 opposite the second plug 20. Preferably, the first plug 10 is free from bubbles or other liquids. However, bubbles and other liquids may be present, provided that particles or the like adsorbing nucleic acids can pass through the first plug 10. Preferably, there are no bubbles or other liquids between the first plug 10 and the second plug 20. However, bubbles and other liquids may be present, provided that particles or the like adsorbing nucleic acids can find passage from the first plug 10 to the second plug 20. Similarly, bubbles and other liquids preferably do not exist between the second plug 20 and the third plug 30. However, bubbles and other liquids may be present, provided that particles or the like adsorbing nucleic acids can find passage from the second plug 20 to the third plug 30.

The fourth plug 40 is provided between the third plug 30 and the fifth plug 50. Another liquid may be disposed on the side of the fifth plug 50 opposite the fourth plug 40. Preferably, the third plug 30 is free from bubbles or other liquids. However, bubbles and other liquids may be present, provided that particles or the like adsorbing nucleic acids can pass through the third plug 30. Preferably, there are no bubbles or other liquids between the third plug 30 and the fourth plug 40. However, bubbles and other liquids may be present, provided that particles or the like adsorbing nucleic acids can find passage from the third plug 30 to the fourth plug 40. Similarly, bubbles and other liquids preferably do not exist between the fourth plug 40 and the fifth plug 50. However, bubbles and other liquids may be present, provided that particles or the like adsorbing nucleic acids can find passage from the fourth plug 40 to the fifth plug 50. Preferably, the fifth plug 50 is free from bubbles or other liquids.

The lengths of the first plug 10, the third plug 30, and the fifth plug 50 along the longitudinal direction of the tube section 100 are not particularly limited, as long as the lengths are sufficient to form the plugs. Specifically, the first plug 10, the third plug 30, and the fifth plug 50 each have a length of 1 mm to 50 mm along the longitudinal direction of the tube section 100. The length is preferably 1 mm to 30 mm, more preferably 5 mm to 20 mm so that particles or the like do not need to move over an excessively long distance. The third plug 30 may have a longer length along the longitudinal direction of the tube section 100. In this way, the second plug 20 does not easily discharge when the fourth plug 40 is adapted to discharge from the end of the tube section 100 on the fifth plug 50 side. Specifically, in this case, the third plug 30 may have a length of 10 mm to 50 mm.

The first plug 10 and the fifth plug 50 serve to prevent exchange of materials between the first washing liquid (second plug 20) or the elution liquid (fourth plug 40) and ambient air such as by evaporation, or external contamination of these liquids when one or both ends of the tube section 100 are open. This makes it possible to maintain the volumes of the first washing liquid and the elution liquid constant, and suppress concentration changes and contamination of these liquids even when one or both ends of the tube section 100 are open to ambient air. This improves the concentration accuracy of nucleic acids or various reagents in nucleic acid extraction.

The third plug 30 serves to suppress mixing of the first washing liquid (second plug 20) and the elution liquid (fourth plug 40). By using a high viscosity oil, the third plug 30 can improve the “wiping effect” of the oil for the particles or the like moving across the interface with the first washing liquid (second plug 20). In this way, the water-soluble components adhering to the particles or the like do not easily enter the third plug 30 (oil) when the particles or the like move into the oil of the third plug 30 from the first washing liquid of the second plug 20.

### 1.3. Second Plug

The second plug 20 is disposed between the first plug 10 and the third plug 30 in the tube section 100. The second plug 20 is formed of a first washing liquid. The first washing liquid is a liquid that is immiscible with both the oil of the first plug 10, and the oil of the third plug 30. The first washing liquid may be, for example, water, or a buffer having a solute concentration of 10 mM or less, preferably 7 mM or less, more preferably 5 mM or less. The buffer composition is not particularly limited, and the buffer may be a tris-HCl buffer, and may contain, for example, EDTA (ethylenediaminetetraacetic acid). Such first washing liquids can efficiently wash the particles or the like adsorbing nucleic acids.

The volume of the second plug 20 is not particularly limited, and may be set as desired by using some index, which may be, for example, the amount of the particles or the like adsorbing nucleic acids. For example, when the volume of the particles or the like is 0.5 μL, the sufficient volume of the second plug 20 is 10 μL or more, preferably 20 μL to 50 μL, more preferably 20 μL to 30 μL. The second plug 20 in these volumes can sufficiently wash the particles or the like when the volume of the particles or the like is 0.5 μL. It is preferable to use the second plug 20 in larger volumes for the washing of the particles or the like. However, the volume of the second plug 20 may be set as desired taking into account factors such as the length and thickness of the tube section 100, and the length of the second plug 20, which varies with the length and thickness of the tube section 100, along the longitudinal direction of the tube section 100.

The second plug 20 maybe configured from a plurality of divided oil plugs. When the second plug 20 is formed of a plurality of divided oil plugs, the second plug 20 includes a plurality of first washing liquid plugs. The second plug 20 with the divided oil plugs is more preferable because, when the washing target is a water-soluble substance, the water-soluble substance can achieve a lower concentration through the divided first washing liquids than when the second plug 20 is of an undivided first washing liquid of the same volume. The second plug 20 may be divided into any number of segments. When the washing target is a water-soluble substance, for example, dividing the second plug 20 into two segments of equal volumes can theoretically lower the concentration of the water-soluble substance to ¼ of the concentration obtained when the second plug 20 is undivided. The number of segments in the second plug 20 may be determined taking into account factors, for example, such as the length of the tube section 100, and the washing target.

### 1.4. Fourth Plug

The fourth plug 40 is disposed in a position between the third plug 30 and the fifth plug 50 in the tube section 100. The fourth plug 40 is formed of an elution liquid.

Here, elution liquid refers to a liquid that desorbs and elutes the adsorbed nucleic acid on the particles or the like into the liquid. Examples of the elution liquid include purified water such as sterile water, distilled water, and ion-exchange water, and an aqueous solution of enzyme, dNTP, probe, primer, and/or buffer dissolved in such water. The elution liquid is a liquid that is immiscible with both the oil of the third plug 30, and the oil of the fifth plug 50.

When the elution liquid is water or an aqueous solution, the nucleic acid adsorbed to the particles or the like can be freed (eluted) by immersing the nucleic acid-adsorbing particles or the like in the elution liquid. When the elution liquid is an aqueous solution dissolving at least one of enzyme, dNTP, probe, primer, and buffer, the components needed for PCR reaction liquid can be contained in the elution liquid either partially or entirely, in addition to freeing (eluting) the nucleic acid adsorbed to the particles or the like. This further reduces the time and labor needed to prepare a PCR reaction liquid with the elution liquid. The concentrations of the enzyme, dNTP, probe, primer, and/or buffer dissolved in the elution liquid of the fourth plug 40 are not particularly limited, and may be set to concentrations as may be desired for the PCR reaction liquid to be prepared.

As used herein, “dNTP” refers to a mixture of four deoxynucleotide triphosphates, dATP (deoxyadenosine triphosphate), dCTP (deoxycytidine triphosphate), dGTP (deoxyguanosine triphosphate), and dTTP (thymidine triphosphate).

The volume of the fourth plug 40 is not particularly limited, and may be set as desired by using some index, which may be, for example, the amount of the particles or the like adsorbing nucleic acids. For example, when the volume of the particles or the like is 0.5 the sufficient volume of the fourth plug 40 is 0.5 μL or more, preferably 0.8 μL to 5 μL, more preferably 1 μL to 3 μL. The fourth plug 40 in these volumes can sufficiently elute nucleic acid from the particles or the like when the volume of the particles or the like is 0.5 μL. For the elution of nucleic acids from the particles or the like, the volume of the fourth plug 40 may be set as desired so as not to overly increase the heat capacity of the reaction liquid, taking into account factors such as the length and thickness of the tube section 100, and the response in the PCR thermal cycle.

### 1.5. Advantages

The nucleic acid extraction device 1000 of the present embodiment has the tube section 100 in which the oil, the first washing liquid, and the elution liquid are disposed in the form of plugs. This makes it possible to extract nucleic acids in a very short time period, by introducing the particles or the like adsorbing nucleic acids into the tube section 100 from the first plug 10 side, and allowing the particles to move into the fourth plug 40. More specifically, the particles or the like adsorbing nucleic acids are introduced into the tube section 100 from the first plug 10 side. The particles are passed through the oil in the first plug 10, and washed with the first washing liquid of the second plug 20. After the passage through the oil in the third plug 30, the nucleic acids can be desorbed from the particles or the like with the elution liquid of the fourth plug 40. Specifically, with the nucleic acid extraction device 1000 of the present embodiment, an elution liquid containing high-purity nucleic acids can be obtained by allowing the particles or the like adsorbing nucleic acids to move through the tube section 100. The nucleic acid extraction device 1000 can thus greatly reduce the time and labor needed for the PCR pretreatment.

### 1.6. Configuration of Nucleic Acid Extraction Device

The nucleic acid extraction device of the present embodiment may be configured to include other functions, in addition to the tube section 100, the first plug 10, the second plug 20, the third plug 30, the fourth plug 40, and the fifth plug 50. The nucleic acid extraction device of the present embodiment may also include a combination of the configurations described below, and modifications of the configurations below.

### 1.6.1. End Portion of Tube Section

FIG. 2 is a diagram schematically representing a nucleic acid extraction device 1010 as a variation of the nucleic acid extraction device. As an example, the nucleic acid extraction device of the present embodiment may have an open end on the fifth plug 50 side of the tube section 100. Specifically, as illustrated in FIG. 2, the nucleic acid extraction device 1010 has an open end on the fifth plug 50 side of the tube section 100. In the nucleic acid extraction device 1010, the fifth plug 50 and the fourth plug 40 can sequentially discharge under the applied pressure from the first plug 10 inside the tube section 100. The nucleic acid extraction device 1010 can thus be used to easily dispense the target nucleic acid-containing elution liquid (fourth plug 40) into, for example, a PCR reaction container.

### 1.6.2. Stopper

FIG. 3 is a diagram schematically representing a nucleic acid extraction device 1020 as another variation of the nucleic acid extraction device. As illustrated in the figure, the nucleic acid extraction device of the present embodiment may further include, for example, a detachable stopper 110 that seals the end of the tube section 100 on the fifth plug 50 side. The stopper 110 may be made of materials, for example, such as rubber, elastomer, and polymer. When the stopper 110 is provided to seal the tube section 100, the stopper 110 may be in contact with the fifth plug 50, or the fifth plug 50 and the stopper 110 may be separated from each other with a gas such as air. The mechanism by which the stopper 110 is made detachable is not particularly limited. In the example of FIG. 3, the stopper 110 is fixed by being partially inserted into the tube section 100. However, the stopper 110 may have a form of a cap.

With the stopper 110 removed, the nucleic acid extraction device 1020 has an open end on the fifth plug 50 side of the tube section 100, and takes the form of the nucleic acid extraction device 1010 shown in FIG. 2. The nucleic acid extraction device 1020 can thus be used to easily dispense the target nucleic acid-containing elution liquid (fourth plug 40) into, for example, a PCR reaction container. With the stopper 110 sealing the end of the tube section 100 on the fifth plug 50 side (the state shown in FIG. 3), the movement of each plug in the tube section 100 can be suppressed. This makes it possible to suppress the plugs from moving with the particles or the like, for example, when the particles or the like are moved inside the tube section 100.

### 1.6.3. Container

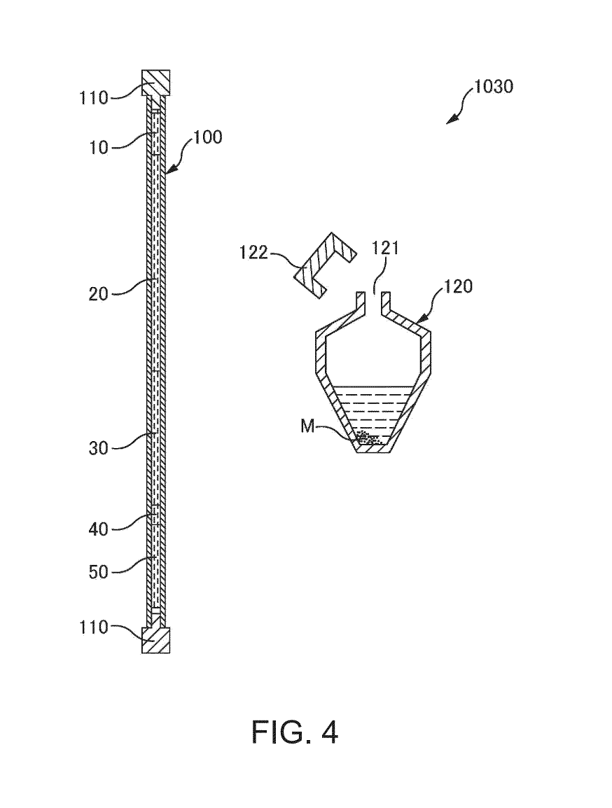

FIG. 4 is a diagram schematically representing a nucleic acid extraction device 1030 as another variation of the nucleic acid extraction device. As illustrated in FIG. 4, the nucleic acid extraction device 1030 has a detachable container 120 that can be joined to the end of the tube section 100 in communication therewith on the first plug 10 side.

The container 120 may be provided as an independent member. The container 120 can contain a liquid inside. The container 120 has an opening 121 through which a liquid or solid can be inserted or removed. In the example of FIG. 4, the opening 121 of the container 120 is joined to the end of the tube section 100 in communication therewith on the first plug 10 side. The container 120 may have a plurality of openings 121. In this case, one of the openings 121 may be joined to the end of the tube section 100 in communication therewith on the first plug 10 side.

The inner volume of the container 120 is not particularly limited, and may be, for example, 0.1 mL to 100 mL. The opening 121 of the container 120 may be structured to be sealable with a lid 122, as desired. The material of the container 120 is not particularly limited, and may be, for example, polymer or metal.

The opening 121 of the container 120 may be joined to the end of the tube section 100 on the first plug 10 side. However, the way the container 120 and the tube section 100 are joined to each other is not particularly limited, provided that the contents do not leak out. With the container 120 and the tube section 100 joined to each other, the container 120 and the tube section 100 are in communication with each other inside. The container 120 maybe detached from the tube section 100, as desired.

By the provision of the container 120 as in the nucleic acid extraction device 1030, for example, the particles or the like, an adsorption liquid, and a specimen can be contained inside the container 120, and the particles or other material can adsorb the nucleic acids in the container 120. The particles or the like can then be easily introduced into the tube section 100 from the first plug 10 side upon joining the container 120 to the end of the tube section 100 on the first plug 10 side.

The adsorption liquid refers to a liquid that provides a medium for the particles (magnetic particles M) to adsorb nucleic acids, and is, for example, an aqueous solution containing a chaotropic agent. The adsorption liquid may contain materials such as a chelating agent, and a surfactant. Specifically, EDTA.2Na or a dihydrate thereof may be dissolved in the adsorption liquid, or the adsorption liquid may contain compounds such as polyoxyethylene sorbitan monolaurate.

The chaotropic agent refers to a substance that weakens the interaction between water molecules, and thereby destabilizes the water molecule structure. Specific examples include guanidium ions, urea, and iodide ions. When the chaotropic agent is present in water, the nucleic acids are adsorbed on the surface of the particles or the like because the nucleic acids in water are thermodynamically more favorable when they exist by being adsorbed on solid than being surrounded by water molecules. Examples of the substances that can generate a chaotropic agent in water include guanidine hydrochloride, and sodium iodide.

The container 120 may be shaken in a state when it is not joined to the tube section 100, and the liquid in the container 120 can be thoroughly stirred by shaking the container 120. This speeds up the adsorption of nucleic acids onto the particles or the like. The container 120 may have a lid 122 for sealing the opening 121. The nucleic acids in a specimen can be quantitatively concentrated in the elution liquid of the fourth plug 40 by varying the amount of the specimen introduced into the container 120, and the liquid volume in the tube section 100 (particularly in the fourth plug 40).

When the material of the container 120 is selected from flexible materials such as rubber, elastomer, and polymer, the container 120 can deform in a state when it is joined to the tube section 100, and increase the pressure inside the tube section 100. This makes it easier to apply pressure from the first plug 10 side of the tube section 100 when discharging the elution liquid of the fourth plug 40 from the end of the tube section 100 on the fifth plug 50 side, enabling the elution liquid to dispense into, for example, a PCR reaction container.

### 1.6.4. Reservoir

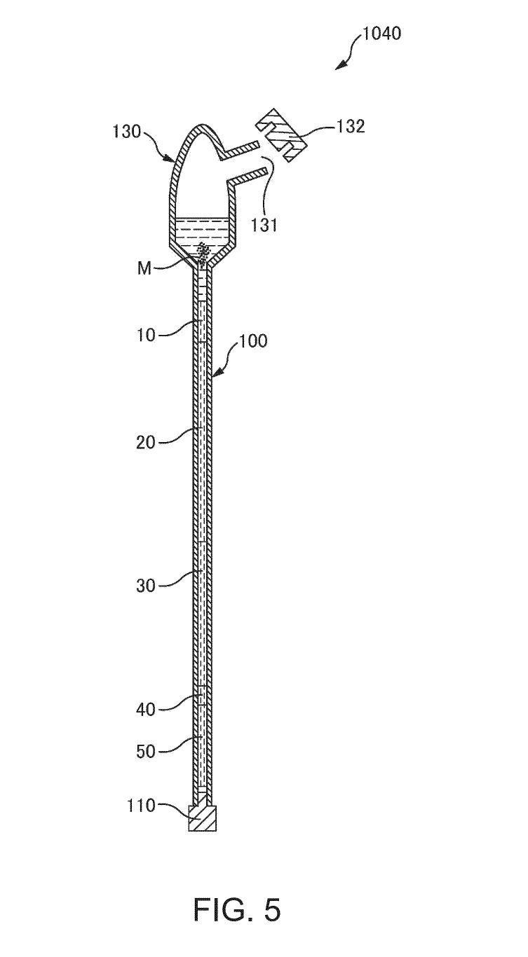

FIG. 5 is a diagram schematically illustrating a nucleic acid extraction device 1040 as an exemplary configuration of the nucleic acid extraction device. As illustrated in FIG. 5, the nucleic acid extraction device 1040 includes a reservoir 130 formed at the end of the tube section 100 on the first plug 10 side in communication with the tube section 100. The reservoir 130 and the tube section 100 are in communication with each other inside.

The reservoir 130 can contain a liquid inside. The reservoir 130 has an opening 131 through which a substance can be introduced into the reservoir 130 from outside. The location of the opening 131 in the reservoir 130 is not particularly limited. The reservoir 130 may have a plurality of openings 131. The inner volume of the reservoir 130 is not particularly limited, and may be, for example, 0.1 mL to 100 mL. The material of the reservoir 130 is not particularly limited, and may be, for example, polymer or metal. The same material used for the tube section 100 may also be used.

By the provision of the reservoir 130 as in the nucleic acid extraction device 1040, for example, the particles or the like, an adsorption liquid, and a specimen can be contained inside the reservoir 130, and the particles or other material can adsorb the nucleic acids in the reservoir 130. The particles or the like can then be easily introduced into the tube section 100 from the first plug 10 side.

The reservoir 130 maybe shaken with the tube section 100, and the liquid in the reservoir 130 can be thoroughly stirred by shaking the reservoir 130. This speeds up the adsorption of nucleic acids onto the particles or the like. The nucleic acids in a specimen can be quantitatively concentrated in the elution liquid by varying the amount of the specimen introduced into the reservoir 130, and the liquid volume in the tube section 100.

When the reservoir 130 is provided as in the nucleic acid extraction device 1040, a detachable lid 132 for sealing the opening 131 of the reservoir 130 may be further provided. When the material of the reservoir 130 is selected from flexible materials such as rubber, elastomer, and polymer, the reservoir 130 can deform in a state when the lid 132 is attached to the reservoir 130, and increase the pressure inside the tube section 100.

This makes it easier to apply pressure from the first plug 10 side of the tube section 100 when discharging the elution liquid of the fourth plug 40 from the end of the tube section 100 on the fifth plug 50 side, enabling performing the procedures from the introduction of a specimen into the container 120 to the dispensing of the elution liquid into, for example, a PCR reaction container. With the lid 132 attached, any leakage as might occur when shaking the reservoir 130 with the tube section 100 can be suppressed, and the particles or the like can adsorb the nucleic acids at improved efficiency.

### 1.6.5. Transport and Storage Structure

An electric field generates between an aqueous solution and the tube when the tube section is touched with hands through electrostatically charged nitrile gloves while carrying or storing the nucleic acid extraction device. In this case, for example, the aqueous solution may be attracted to the tube inner wall, and adhere thereto when being pushed out of the tube in the manner described below. This may disrupt the plug as the applied force pushes only the oil while the aqueous solution remains still, or may cause droplets of the aqueous solution to float in the oil as the aqueous solution is repelled by the tube inner wall. The disrupted aqueous solution, or droplets of the aqueous solution move in the oil under static electricity, and may mix with pretreatment reagents in other plugs. This changes the composition of the aqueous solution in the mixed plug, and the plug may no longer remain functional.

The nucleic acid extraction device thus preferably has a structure with which the oil or the aqueous solution in the tube section can be prevented from being electrified during transport or storage, or a structure that keeps the oil or aqueous solution in the tube section away from a charged substance such as hands wearing electrostatically charged nitrile gloves. As used herein, “preventing electrification” does not necessarily mean completely eliminating the charge, but is intended to mean that the generated charge is reduced to such an extent that the tube can sufficiently function.

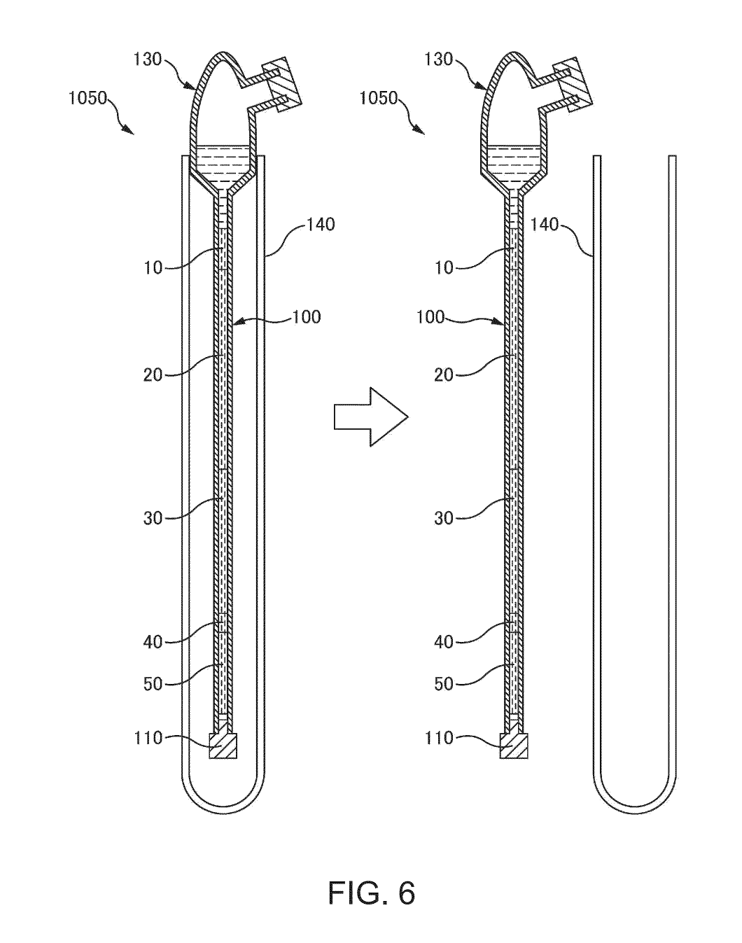

The oil or aqueous solution in the tube section can be kept away from a charged substance such as hands wearing electrostatically charged nitrile gloves, for example, by providing a cover section that surrounds the tube section. FIG. 6 is a diagram schematically illustrating a nucleic acid extraction device 1050 with a cap 140 provided as a detachable cover section for covering the tube section 100. The nucleic acid extraction device 1050 is shown with the cap 140 being attached to the device, and removed from the device. The cap 140 may be made of materials, for example, such as non-magnetic metal, glass, plastic, rubber, and stone. For nucleic acid extraction with the nucleic acid extraction device 1040, a permanent magnet 410 (described later) may be brought close to the tube section 100 after removing the cap 140.

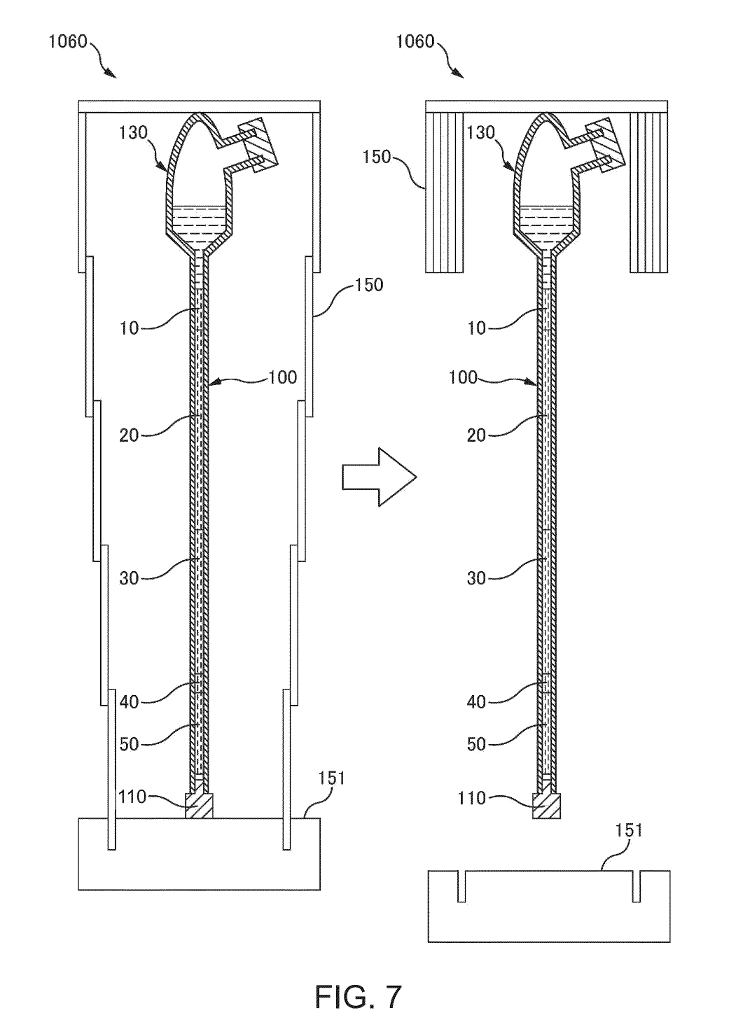

FIG. 7 is a diagram schematically illustrating a nucleic acid extraction device 1060 with an expandable cap 150 having a lid 151, the expandable cap 150 being provided as a cover section for covering the tube section 100. The nucleic acid extraction device 1060 is shown with the expandable cap 150 being attached to the device, and in a compressed form. The expandable cap 150 may be any structure, as long as it can expand and compress along the direction of extension of the tube section 100, and may have, for example, an accordion structure. For nucleic acid extraction with the nucleic acid extraction device 1060, the lid 151 is removed, and the expandable cap 150 is compressed along the direction of extension of the tube section 100 to expose the tube section 100. The expandable cap 150 may be compressed by hand. Alternatively, a device insertion opening of a diameter smaller than the outer diameter of the expandable cap 150 may be provided in the nucleic acid extraction apparatus, and the expandable cap 150 may be compressed by pushing the nucleic acid extraction device 1060 into the device insertion opening. The permanent magnet 410 (described later) may then be brought close to the tube section 100.

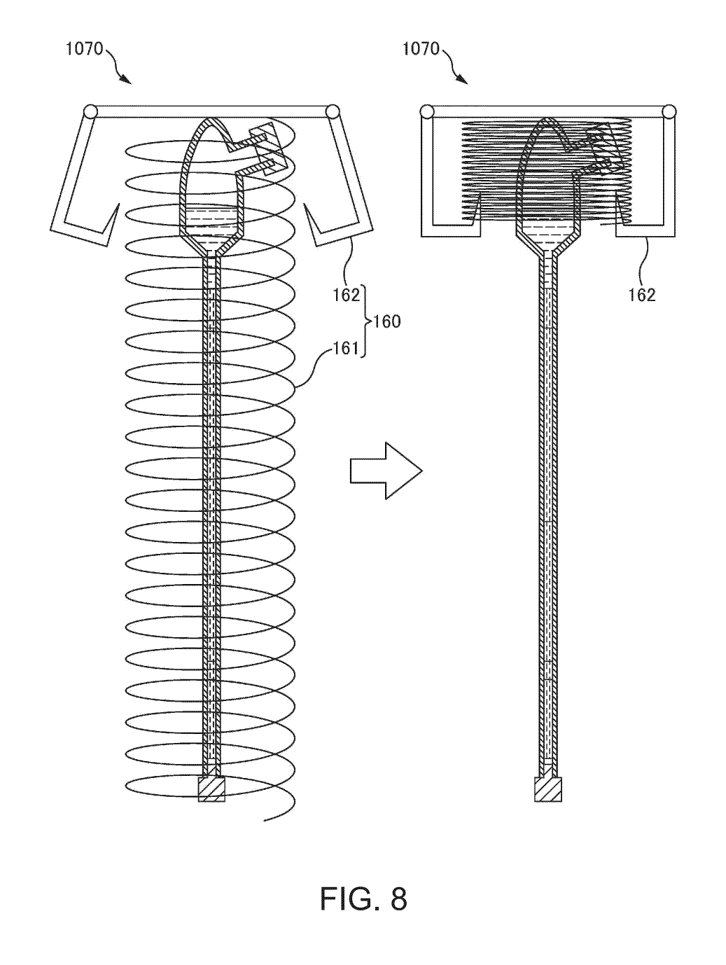

FIG. 8 is a diagram schematically illustrating a nucleic acid extraction device 1070 with a cover 160 having a spring 161 and a holder 162 for the spring 161, the cover 160 being provided as an expandable cover section for covering the tube section 100. The nucleic acid extraction device 1070 is shown with the cover 160 being attached to the device with the spring 161 stretched and compressed. When the spring 161 tends to swing sideways, a pillar for fixing the spring 161 at a predetermined position may be provided inside the spring 161. For nucleic acid extraction with the nucleic acid extraction device 1070, the spring 161 is compressed by hand along the direction of extension of the tube section 100 to expose the tube section 100, and the compressed spring 161 is fixed to the holder 162. Alternatively, a device insertion opening of a diameter smaller than the outer diameter of the spring 161 may be provided in the nucleic acid extraction apparatus, and the spring 161 may be compressed to expose the tube section 100 by pushing the device into the device insertion opening. The permanent magnet 410 (described later) may then be brought close to the tube section 100.

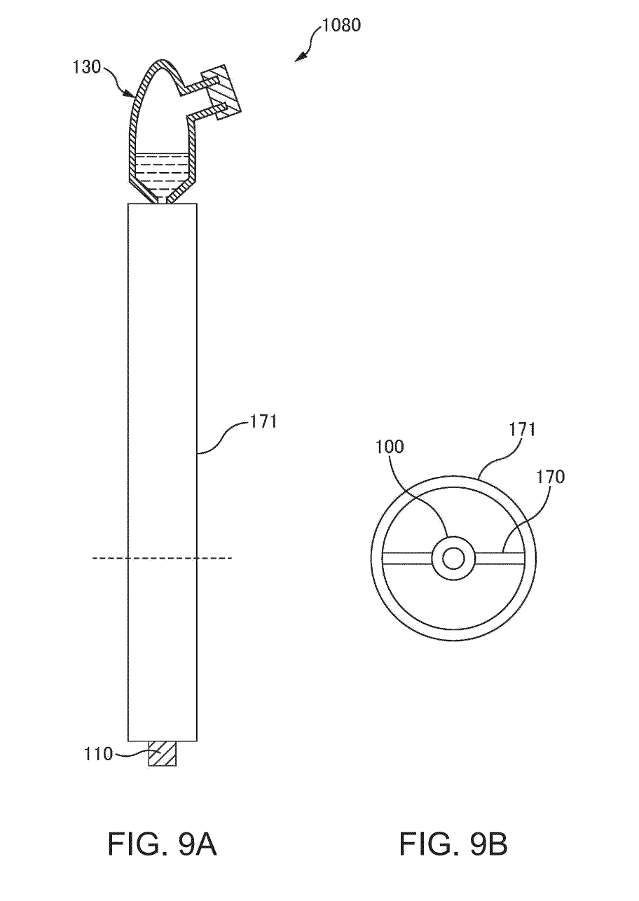

FIG. 9A is a diagram schematically illustrating a nucleic acid extraction device 1080 with a cover section 171 formed on the tube section 100 via a supporting section 170. FIG. 9B is a cross sectional view at the broken line shown in FIG. 9A. For nucleic acid extraction with the nucleic acid extraction device 1080, the permanent magnet 410 may be brought close to the tube section 100 over the cover section 171.

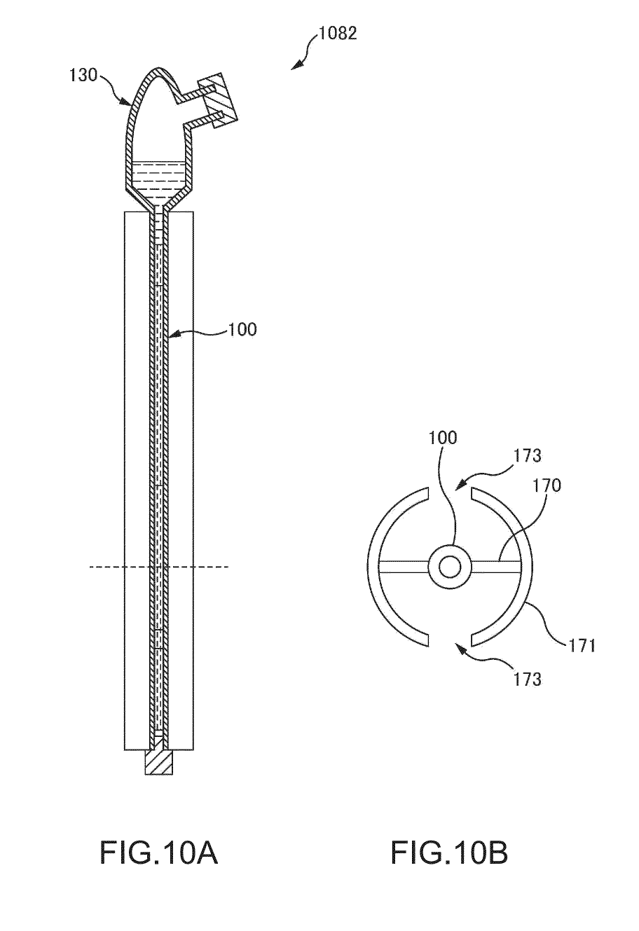

FIG. 10A is a diagram schematically illustrating a nucleic acid extraction device 1082 with slits 173 formed in the cover section 171 and that extend along the direction of extension of the tube section 100. FIG. 10B is a cross sectional view at the broken line shown in FIG. 10A. For nucleic acid extraction with the nucleic acid extraction device 1082, the permanent magnet 410 (described later) may be brought close to the tube section 100 by being inserted into the slits 173.

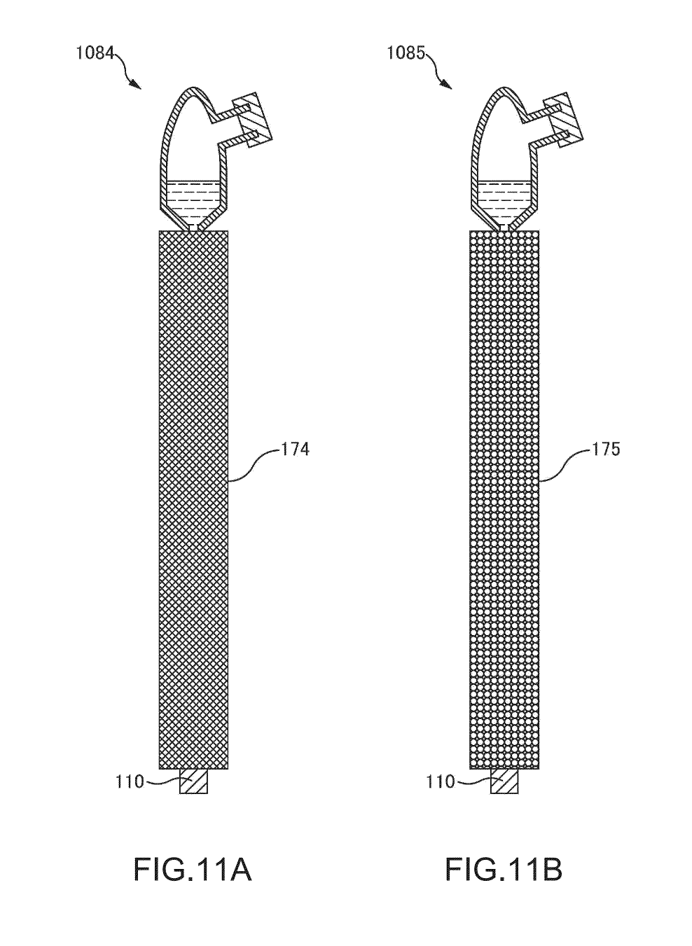

FIG. 11A is a diagram schematically representing a nucleic acid extraction device 1084 with a cover section 174 having a mesh structure. FIG. 11B is a diagram schematically representing a nucleic acid extraction device 1085 with a cover section 175 having dot-like holes (perforations). Despite the one or more holes provided in the cover sections of the nucleic acid extraction devices 1084 and 1085, the hole size is smaller than the size of a finger, and it is unlikely that the finger of a person holding and carrying the nucleic acid extraction devices 1084 and 1085 comes close to the tube section 100 over the cover section.

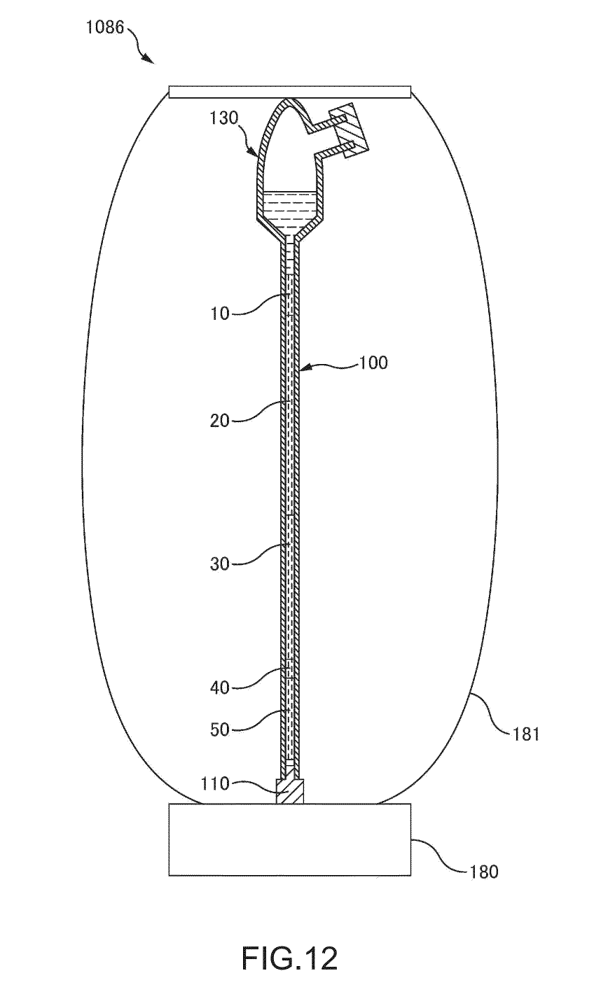

FIG. 12 is a diagram schematically illustrating a nucleic acid extraction device 1086 placed in a bag 181 attached to a clamp 180. Preferably, the bag 181 is inflated by sealing a gas such as nitrogen so that hands holding the bag 181 with electrostatically charged nitrile gloves while carrying the nucleic acid extraction device 1086 do not come any closer than 3 mm from the inner cavity surface of the tube section 100, and prevent the tube section from adhering to the cover section. The material of the bag 181 is not particularly limited, as long as it is deformable, and may be, for example, vinyl. For nucleic acid extraction with the nucleic acid extraction device 1086, the bag 181 may be torn to expose the tube section 100. In this way, the permanent magnet 410 can be brought close to the tube section 100. The bag 181 may be structured in such a way that it easily rips upon pulling the clamp 180.

The side wall thickness of the tube section of the nucleic acid extraction device may be increased so that a charged substance such as hands wearing electrostatically charged nitrile gloves do not come close to the inner cavity of the tube section. In this way, the oil or aqueous solution in the tube section can be prevented from being electrified by hands or other such electrostatically charged object touching the tube section. The side wall of the tube section may have any thickness, provided that it can prevent the electrification of the oil or aqueous solution in the inner cavity of the tube section, and that the side wall of the tube section allows for the permanent magnet operation (described later). The side wall thickness of the tube section is preferably 3 mm to 9.5 mm.

Materials such as non-magnetic conductive metals and metal alloys may be embedded in the side wall of the tube section to further improve the electrification preventing effect for the oil or aqueous solution in the tube section. For example, the side wall of the tube section may be embedded with a coiled copper wire.

The plugs in the tube section can be stably maintained in position by preventing the electrification of the oil or aqueous solution in the tube section in the manner described above. Preventing the electrification of the nucleic acid extraction device makes it easier to automate the nucleic acid extraction with the nucleic acid extraction device.

### 1.6.6. Sixth Plug and Seventh Plug

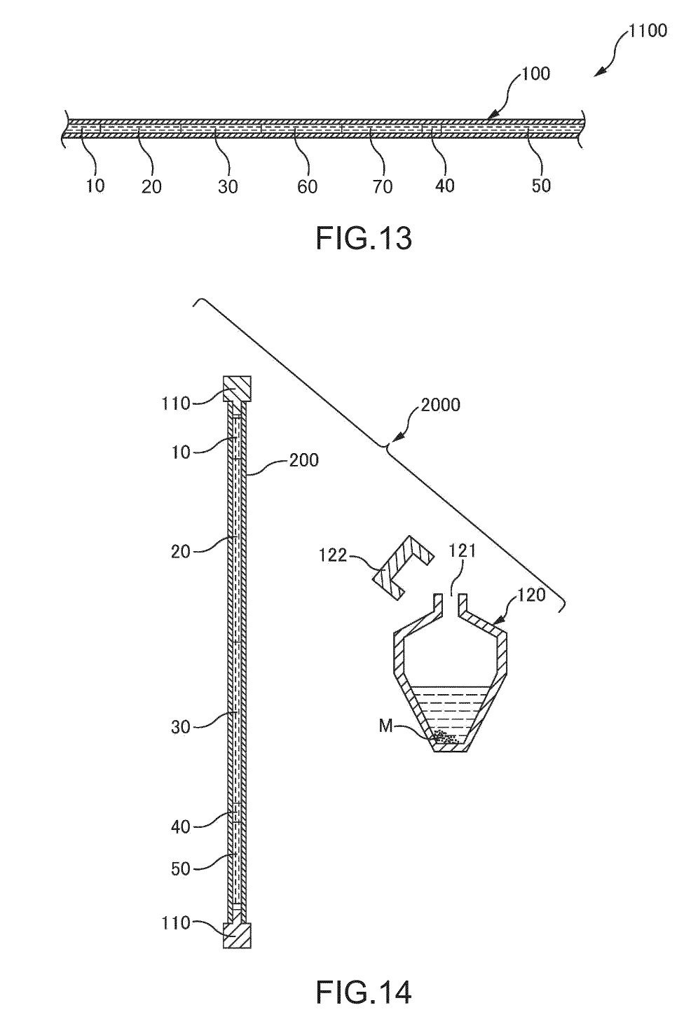

The nucleic acid extraction device of the present embodiment may have a sixth plug and a seventh plug inside the tube section. FIG. 13 is a diagram schematically illustrating a nucleic acid extraction device 1100 having a sixth plug 60 and a seventh plug 70 inside the tube section 100.

The nucleic acid extraction device 1100 is configured so that the sixth plug 60 formed of a second washing liquid immiscible with oil, and the seventh plug 70 formed of oil are additionally provided between the third plug 30 and the fourth plug 40 inside the tube section 100 of the nucleic acid extraction device, in this order from the third plug 30 side.

The sixth plug 60 is positioned on the side of the third plug 30 opposite the second plug 20 in the tube section 100. The sixth plug 60 is formed of a second washing liquid. The second washing liquid is a liquid that is immiscible with both the oil of the third plug 30, and the oil of the seventh plug 70. The second washing liquid may be water, or a buffer with a solute concentration of 10 mM or less, preferably 7 mM or less, more preferably 5 mM or less. The buffer composition is not particularly limited, and the buffer may be a tris-HCl buffer, and may contain EDTA (ethylenediaminetetraacetic acid) or the like. The composition of the second washing liquid may be the same as or different from the composition of the first washing liquid.

The volume of the sixth plug 60 is not particularly limited, and may be set as desired by using some index, which may be, for example, the amount of the particles or the like adsorbing nucleic acids. For example, when the volume of the particles or the like is 0.5 μL, the sufficient volume of the sixth plug 60 is 10 μL or more, preferably 20 μL to 50 μL, more preferably 20 μL, to 30 μL. The sixth plug 60 in these volumes can sufficiently wash the particles or the like when the volume of the particles or the like is 0.5 μL. It is preferable to use the sixth plug 60 in larger volumes for the washing of the particles or the like. However, the volume of the sixth plug 60 may be set as desired taking into account factors such as the length and thickness of the tube section 100, and the length of the sixth plug 60, which varies with the length and thickness of the tube section 100, along the longitudinal direction of the tube section 100.

The sixth plug 60 may be configured from a plurality of divided oil plugs. When the sixth plug 60 is formed of a plurality of divided oil plugs, the sixth plug 60 includes a plurality of second washing liquid plugs. The sixth plug 60 with the divided oil plugs is more preferable because, when the washing target is a water-soluble substance, the water-soluble substance can achieve a lower concentration through the divided second washing liquids than when the sixth plug 60 is of an undivided second washing liquid of the same volume. The sixth plug 60 may be divided into any number of segments. When the washing target is a water-soluble substance, for example, dividing the sixth plug 60 into two segments of equal volumes can theoretically lower the concentration of the water-soluble substance to ¼ of the concentration obtained when the sixth plug 60 is undivided. The number of segments in the sixth plug 60 maybe set as desired taking into account factors, for example, such as the length of the tube section 100, and the washing target. When the same liquid is used for the first washing liquid of the second plug 20 and the second washing liquid of the sixth plug 60, the same effect can be obtained that results from dividing the second plug 20 in the nucleic acid extraction device that does not include the sixth plug 60 or the seventh plug 70.

The seventh plug 70 is formed of an oil that is immiscible with the liquids of the adjacent sixth plug 60 and fourth plug 40. The oil of the seventh plug 70 may be different from the oils of the first plug 10, the third plug 30, and the fifth plug 50. The oil may be selected from the oils exemplified above for the first plug 10 and other plugs.

Preferably, the seventh plug 70 is free from bubbles or other liquids. However, bubbles and other liquids may be present, provided that particles or the like adsorbing nucleic acids can pass through the seventh plug 70. Preferably, there are no bubbles or other liquids between the seventh plug 70 and the adjacent fourth and sixth plugs 40 and 60. However, bubbles and other liquids may be present, provided that particles or the like adsorbing nucleic acids can move inside the tube section 100. Preferably, the seventh plug 70 is free from bubbles or other liquids.

The length of the seventh plug 70 along the longitudinal direction of the tube section 100 is not particularly limited, as long as the length is sufficient to form the plug. Specifically, the length of the seventh plug 70 along the longitudinal direction of the tube section 100 is 1 mm to 50 mm, and is preferably 1 mm to 30 mm, more preferably 5 mm to 20 mm so that particles or the like do not need to move over an excessively long distance. The seventh plug 70 may have a longer length along the longitudinal direction of the tube section 100 in the nucleic acid extraction device 1100. In this way, the sixth plug 60 does not easily discharge when the fourth plug 40 is adapted to discharge through the tube section 100 at the end of the fifth plug 50. Specifically, in this case, the seventh plug 70 may have a length of 10 mm to 50 mm.

The seventh plug 70 serves to suppress mixing of the second washing liquid (sixth plug 60) and the elution liquid (fourth plug 40). By using a high viscosity oil, the seventh plug 70 can improve the “wiping effect” of the oil for the particles or the like moving across the interface with the second washing liquid (sixth plug 60). In this way, the water-soluble components adhering to the particles or the like do not easily enter the seventh plug 70 (oil) when the particles or the like move into the oil of the seventh plug 70 from the second washing liquid of the sixth plug 60.

The nucleic acid extraction device 1100 enables washing the nucleic acid-adsorbing particles or the like in the second plug 20 and the sixth plug 60. This can further improve the efficiency of washing the particles and other materials.

The first washing liquid of the second plug 20 in the nucleic acid extraction device 1100 may contain a chaotropic agent. For example, by containing guanidine hydrochloride in the first washing liquid of the second plug 20, the second plug 20 can wash the particles or the like while maintaining or strengthening the adsorption of the nucleic acids by the particles or the like. When the second plug 20 contains guanidine hydrochloride, the concentration of the guanidine hydrochloride maybe, for example, 3 mol/L to 10 mol/L, preferably 5 mol/L to 8 mol/L. With this guanidine hydrochloride concentration range, other foreign substances can be washed while stably maintaining the nucleic acids adsorbed to the particles or the like.

Because the second washing liquid of the sixth plug 60 is water or buffer, the nucleic acids adsorbed to the particles or the like can be more stably maintained while washing the particles or the like in the second plug 20 (first washing liquid), and the sixth plug 60 (second washing liquid) can further wash the particles or the like while diluting the chaotropic agent.

It is to be understood that the additional components, including the stopper, the container, and the reservoir described above may be provided for the configuration of the nucleic acid extraction device 1100 having the sixth plug 60 and the seventh plug 70 inside the tube section 100, and that the same effect can be obtained by the provision of these components.

### 2. NUCLEIC ACID EXTRACTION KIT

FIG. 14 is a schematic diagram representing an example of the nucleic acid extraction kit of the present embodiment. The nucleic acid extraction kit 2000 illustrated in FIG. 14 includes the components forming the nucleic acid extraction device described above. The configurations described in conjunction with the nucleic acid extraction device in the foregoing Section 1 will be referred to by using the same reference numerals, and will not be described in detail.

The nucleic acid extraction kit 2000 of the present embodiment includes a tube 200 and a container 120. The tube 200 includes the first plug 10 formed of oil, the second plug 20 formed of a first washing liquid immiscible with oil, the third plug 30 formed of oil, the fourth plug 40 formed of an elution liquid that is immiscible with oil, and the fifth plug 50 formed of oil, disposed in this order inside the tube. The container 120 can be joined to the end of the tube 200 on the first plug 10 side in communication with the inside of the tube.

The tube 200 represents the tube section 100 of the nucleic acid extraction device 1000 with open ends. The tube 200 is cylindrical in shape, and has an inner cavity that allows for passage of a liquid along the longitudinal direction. The attributes of the tube 200, including the inner shape, outer shape, size, properties, and material are as described for the tube section 100 of the nucleic acid extraction device 1000. The same plugs disposed in the tube section 100 of the nucleic acid extraction device 1000 are disposed in the tube 200. The ends of the tube 200 may be sealed with detachable stoppers 110. With the ends of the tube 200 sealed with the stoppers 110, for example, the nucleic acid extraction kit 2000 can be stored and transferred more easily. With the stopper 110 sealing the end of the tube 200 on the fifth plug 50 side during use, the movement of each plug in the tube 200 can be suppressed when the particles or the like are moved inside the tube 200. This makes the washing and extraction even easier. Because the stopper 110 is detachable, the tube 200 can have an open end on the fifth plug 50 side, and the elution liquid of the fourth plug 40 that has eluted the nucleic acids can easily discharge from the end of the tube 200 on the fifth plug 50 side.

The container 120 is the same container 120 described in conjunction with the nucleic acid extraction device 1000.

In the example of FIG. 14, the ends of the tube 200 are sealed with the detachable stoppers 110. The nucleic acid extraction kit 2000 may include a lid 122 for detachably sealing the opening 121 of the container 120, and the opening 121 of the container 120 may be sealed with the detachable lid 122. In the nucleic acid extraction kit 2000, the container 120 may contain some of or all of the adsorption liquid components.

In the nucleic acid extraction kit 2000, the container 120 may contain the adsorption liquid and magnetic particles. In this way, the adsorption of the specimen nucleic acids by the magnetic particles can take place in the container 120 upon introducing the specimen into the container 120. This makes it possible to quickly perform the PCR pretreatment, without using other containers. The opening 121 of the container 120 may be sealed with the detachable lid 122, as desired. The magnetic particles will be described later in detail.

As described above, when the material of the container 120 is a flexible material, the container 120 can deform in a state when it is joined to the tube 200, and increase the pressure inside the tube 200. This makes it easier to apply pressure from the first plug 10 side of the tube 200 when discharging the elution liquid of the fourth plug 40 from the end of the tube section 100 on the fifth plug 50 side, enabling the elution liquid to easily dispense into, for example, a PCR reaction container.

The nucleic acid extraction kit 2000 may be configured to include other members, for example, such as a stopper, a lid, a user's manual, reagents, and a case, in addition to the tube 200 and the container 120. The tube 200 has been described as containing five plugs. However, it is to be understood that the tube 200 (tube section 100) may contain other plugs, including the sixth plug 60 and the seventh plug 70, as desired, as described in conjunction with the nucleic acid extraction device in the foregoing Section 1.6.

The nucleic acid extraction kit 2000 of the present embodiment has the container 120 that can be joined to the end of the tube 200 on the first plug 10 side in communication with the inside of the tube. The adsorption of nucleic acids by particles or the like can thus take place upon containing particles or the like and a specimen in the container 120. Further, the particles or the like can easily be introduced into the tube 200 from the first plug side by joining the container 120 to the end of the tube 200 on the first plug 10 side. Because the nucleic acid extraction kit 2000 of the present embodiment has the container 120, the container 120 can be shaken to thoroughly stir the liquid inside the container 120. This makes it possible to more quickly adsorb the nucleic acids to the particles or the like.

With the container 120 joined to the tube 200, the particles or the like adsorbing the nucleic acids can be easily introduced into the tube 200 on the first plug 10 side, and moved to the fourth plug 40. This makes it possible to easily perform nucleic acid extraction in a very short time period. The nucleic acid extraction kit 2000, by moving the nucleic acid-adsorbing particles or the like inside the tube 200, can produce an elution liquid containing the nucleic acids in high purity. The nucleic acid extraction kit 2000 can thus greatly reduce the time and labor required for the PCR pretreatment.

### 3. NUCLEIC ACID EXTRACTION METHOD

The nucleic acid extraction device, the nucleic acid extraction kit, modifications of these, and the nucleic acid extraction apparatus (described later) all can be preferably used for the nucleic acid extraction method of the present embodiment. The following exemplary description of the nucleic acid extraction method is based on the nucleic acid extraction kit 2000.

The nucleic acid extraction method of the present embodiment includes introducing a nucleic acid-containing specimen into a flexible container 120 containing magnetic particles M and an adsorption liquid, swinging the container 120 to adsorb the nucleic acids to the magnetic particles M, joining the container 120 to the end of the tube 200 on the first plug 10 side with the inside of the container 120 in communication with the inside of the tube 200 in which the first plug 10 formed of oil, the second plug 20 formed of a first washing liquid immiscible with oil, the third plug 30 formed of oil, the fourth plug 40 formed of an elution liquid immiscible with oil, and the fifth plug 50 formed of oil are disposed in this order, moving the magnetic particles M from inside of the container 120 to the position of the fifth plug 50 through the tube 200 under applied magnetic force, and eluting the nucleic acids from the magnetic particles M into the elution liquid of the fourth plug 40.

Various particles (for example, silica particles, polymer particles, and magnetic particles) may be used in the nucleic acid extraction method of the present embodiment, provided that the particles can adsorb nucleic acids with an adsorption liquid, and can move inside the tube 200. The magnetic particles M used in the following embodiment of the nucleic acid extraction method are magnetic material-containing particles that can adsorb nucleic acids on the particle surface. When particles or the like other than magnetic particles M are to be moved inside the tube, for example, these may be moved by making use of gravity or potential difference.

In the nucleic acid extraction method of the present embodiment, materials that pass magnetic force are selected for the container 120 and the tube 200, and a magnetic force is externally applied to the container 120 and the tube 200 to move the magnetic particles M inside the container 120 and the tube 200.

The specimen contains the nucleic acid of interest. This will also be referred to simply as “target nucleic acid.” The target nucleic acid is, for example, DNA and/or RNA (DNA: deoxyribonucleic acid and/or RNA: ribonucleic acid). The target nucleic acid is extracted from the specimen, and eluted into an elution liquid by using the nucleic acid extraction method of the present embodiment. The nucleic acid can then be used, for example, as a template for PCR. Examples of the specimen include various biological samples such as, blood, sinus mucus, and oral mucosa.

### 3.1. Introducing Specimen into Container

The step of introducing a specimen into the container 120 may be performed, for example, by contacting a cotton swab to a specimen, and immersing the cotton swab into the adsorption liquid through the opening 121 of the container 120. The specimen may be introduced through the opening 121 of the container 120 with an instrument such as a pipette. When the specimen is a paste or solid form, for example, the specimen may be contacted to the inner wall of the container 120, or charged into the container 120 through the opening 121 of the container 120, using an instrument such as a spoon and tweezers.

### 3.2. Adsorbing Nucleic Acids to Magnetic Particles

The step of adsorbing nucleic acids is performed by swinging the container 120. This step can be performed more efficiently when the container 120 is sealed with the lid 122 that seals the opening 121 of the container 120. In this step, the target nucleic acid adsorbs to the surfaces of the magnetic particles M under the effect of a chaotropic agent. In this step, nucleic acids other than the target nucleic acid, and proteins may adsorb to the surfaces of the magnetic particles M, in addition to the target nucleic acid.

The container 120 may be swung with a device such as a vortex shaker, or with a hand of an operator. The container 120 may be swung under the externally applied magnetic field, using the magnetism of the magnetic particles M. The swing duration of the container 120 may be set as desired, and simply swinging the container 120 by hand for 10 seconds is sufficient to stir the contents and adsorb the nucleic acids to the surfaces of the magnetic particles M, for example, when the container 120 is cylindrical in shape with a diameter of about 20 mm and a height of about 30 mm.

### 3.3. Joining Container to Tube

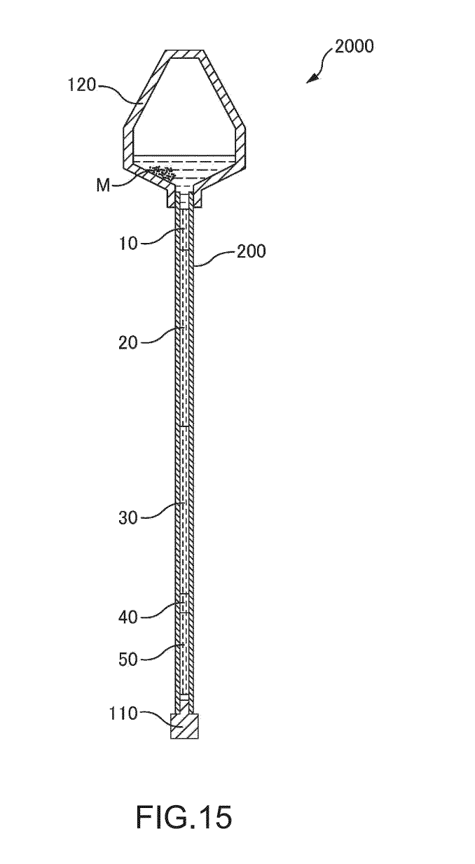

As illustrated in FIG. 15, the container 120 is joined to the end of the tube 200 on the first plug 10 side. With the stopper 110 attached to the seventh plug 70 side, the plugs in the tube 200 do not easily move inside the tube 200 even when the stopper 110 on the side of the first plug 10 is removed. This step is performed after removing the stopper 110, when it is attached to the end of the tube 200 on the first plug 10 side. The container 120 and the tube 200 are joined to each other in a manner that prevents leaking of the contents, and are brought in communication with each other to allow for passage of the contents inside the container 120 and the tube 200.

### 3.4. Moving Magnetic Particles

After the foregoing steps, the magnetic particles M that have adsorbed the nucleic acids inside the container 120 are in condition for passage through the tube 200. The magnetic particles M adsorbing the nucleic acids may be guided into the tube 200 by making use of gravity or centrifugal force; however, the method is not particularly limited. In the present embodiment, this is performed by applying a magnetic force from outside of the container 120 and the tube 200. A magnetic force may be applied, for example, with a permanent magnet or an electromagnet. It is however more preferable to use a permanent magnet because it does not produce heat. When using a permanent magnet, the magnet may be moved with a hand of an operator, or with a device such as a mechanical device. The magnetic particles M have the property to be attracted under a magnetic force. By taking advantage of this property, the magnetic particles M are moved into the tube 200 from the container 120 by varying the relative positions of the container 120 and the tube 200 with respect to the permanent magnet. This moves the magnetic particles M from the first plug 10 to the fourth plug 40 through the series of plugs. The time it takes for the magnetic particles M to pass through each plug is not particularly limited, and the magnetic particles M may be moved back and forth within the same plug along the longitudinal direction of the tube 200.

### 3.5. Eluting Nucleic Acids

Upon the magnetic particles M reaching the fourth plug 40, the nucleic acids adsorbed to the magnetic particles M elute into the elution liquid of the fourth plug 40 under the effect of the elution liquid. This step completes the elution of the nucleic acids into the elution liquid, and the extraction of the nucleic acids from the specimen.

### 3.6. Advantages

The nucleic acid extraction method of the present embodiment enables performing nucleic acid extraction with ease in a very short time period. The nucleic acid extraction method of the present embodiment moves the nucleic acid-adsorbing magnetic particles M in the tube 200, and can produce an elution liquid containing nucleic acids in high purity. The nucleic acid extraction method of the present embodiment can greatly reduce the time and labor required for the PCR pretreatment.

### 3.7. Discharging Fourth Plug from Tube

The nucleic acid extraction method of the present embodiment may include the step of deforming the container 120, and discharging the fifth plug 50 and the fourth plug 40 from the end of the tube 200 opposite the end joined to the container 120.

This step may be performed by deforming the container 120 after the step of eluting nucleic acids described in Section 3.5 above. The discharge of the fourth plug 40 follows the discharge of the fifth plug 50. The stopper 110 sealing the tube 200 on the fifth plug 50 side is removed to open this side of the tube before performing the step.

Deforming the container 120 under external force and increasing the inner pressure moves each plug from the first plug 10 side to the fifth plug 50 side of the tube 200 under the exerted pressure. This discharges the fifth plug 50 and the fourth plug 40, in this order, from the end of the tube 200 on the fifth plug 50 side. Here, the third plug 30 (or the seventh plug 70) may discharge; however, the second plug 20 (or the sixth plug 60) must not discharge. The discharge of the second plug 20 (or the sixth plug 60) can easily be prevented, for example, by setting a larger volume for the third plug 30 (or the seventh plug 70) than for the other plugs, and increasing the length of the third plug 30 (or the seventh plug 70) along the longitudinal direction of the tube 200.

The fourth plug 40 and the fifth plug 50 discharge into, for example, a PCR reaction container. That is, both the elution liquid and the oil are dispensed into the PCR reaction container. However, because the oil typically does not affect PCR reactions, for example, the oil of the same kind used for the fifth plug 50 may be contained in the PCR reaction container beforehand. In this case, the elution liquid containing the target nucleic acid can be introduced into the PCR reaction container without contacting the target nucleic acid-containing elution liquid to ambient air when the step is performed with the tip of the tube 200 being submerged in the oil. The nucleic acid extraction method of the present embodiment, with this step, can easily dispense the target nucleic acid-containing elution liquid into, for example, a PCR reaction container.

### 3.8. Variations

### 3.8.1. Variation of the Step of Moving Magnetic Particles

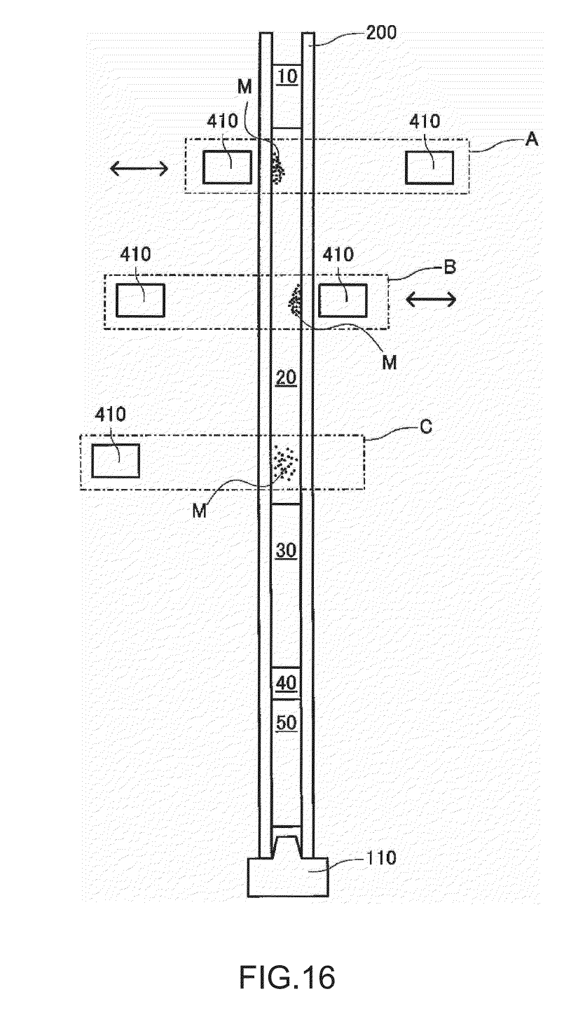

FIG. 16 is a schematic diagram explaining a variation of the nucleic acid extraction method of the present embodiment.

The step of moving magnetic particles in the foregoing Section 3.4 was described through the case where a magnetic force is externally applied to the magnetic particles M to move the magnetic particles M through the plugs from the first plug 10 to the fourth plug 40. However, the magnetic particles M upon being moved to the second plug 20 maybe caused to vibrate, or to repeatedly undergo diffusion and aggregation in the second plug 20 by varying the externally applied magnetic force. In this way, the washing effect for the magnetic particles M by the first washing liquid of the second plug 20 can improve.

Specifically, as indicated by A and B in FIG. 16, when a pair of permanent magnets 410 is used as a means to apply magnetic force, the permanent magnets 410 move the magnetic particles M out of the container 120, and, upon the magnetic particles M reaching the second plug 20 through the first plug 10, one of the permanent magnets 410 is moved away from the tube 200 while the other permanent magnet 410 approaches the tube from the opposite side. This makes it possible to vibrate the magnetic particles M in the second plug 20 in directions orthogonal to the longitudinal direction of the tube 200 (repeating the movement indicated by A and B in the figure). In this way, the washing effect for the magnetic particles M by the first washing liquid of the second plug 20 can improve. The magnetic particles M may also be washed in this manner with a plurality of second plugs 20, and/or with the sixth plugs 60, when the second plug 20 is divided or when the sixth plug 60 is provided in the tube 200.

As indicated by C in FIG. 16, the magnetic particles M can diffuse in the second plug 20 when the permanent magnet 410 is simply moved away from the tube 200. This is possible because the magnetic particles M, by being hydrophilic on the surface, do not easily enter the oils of the first plug 10 and the third plug 30 even when, for example, diffusion occurs in the second plug 20 under the reduced magnetic force.

Specifically, the permanent magnet 410 is used to move the magnetic particles M out of the container 120, and the permanent magnet 410 is moved away from the tube 200 to diffuse the magnetic particles M in the second plug 20 upon the magnetic particles M reaching the second plug 20 through the first plug 10. The magnetic particles M can then be moved and guided to the fourth plug 40 through the third plug 30 under the magnetic force of the permanent magnet 410.

The embodiment in which the magnetic particles M are caused to vibrate, or to repeatedly undergo diffusion and aggregation by varying the externally applied magnetic force is also applicable in the state where the magnetic particles M are present in the adsorption liquid in the container 120, or in the fourth plug 40 (elution liquid).

### 3.8.2. Variation of the Step of Eluting Nucleic Acids

The step of eluting nucleic acids as described in the foregoing Section 3.5 may be performed by heating the fourth plug 40. Examples of the method for heating the fourth plug 40 include contacting a heat medium such as a heat block to a position corresponding to the fourth plug 40 of the tube 200, a method using a heat source such as a heater, and electromagnetic heating.

When heating the fourth plug 40, the plugs other than the fourth plug 40 may also be heated. It is, however, preferable not to heat the other plugs when the magnetic particles M adsorbing the nucleic acids are present in the washing liquid plugs. The temperature that reaches upon heating the fourth plug 40 is preferably 35° C. to 85° C., more preferably 40° C. to 80° C., further preferably 45° C. to 75° C. in terms of elution efficiency, and in terms of suppressing enzyme deactivation when the elution liquid contains enzymes for PCR.

By heating the fourth plug 40 in the step of eluting nucleic acids, the nucleic acids adsorbed to the magnetic particles M can more efficiently elute into the elution liquid. Further, the nucleic acids that did not elute into the washing liquid and remain adsorbed to the magnetic particles M can elute into the elution liquid even when the first washing liquid or the second washing liquid has the same or similar composition to the composition of the elution liquid. That is, the nucleic acids can further elute into the elution liquid after the nucleic acid-adsorbing magnetic particles M are washed with the first washing liquid or the second washing liquid. In this way, it is ensured that the magnetic particles M are sufficiently washed, and that the elution into the elution liquid occurs at sufficient concentrations even when the washing liquid composition and the elution liquid composition are the same or similar.

3.8.3. Variation of the Step of Discharging Fourth Plug from Tube

When the method includes the step of discharging the fourth plug from the tube as described in the foregoing Section 3.7, the fourth plug 40 to be discharged in this step may contain the magnetic particles M that remain after the elution of the adsorbed nucleic acids into the elution liquid. Alternatively, the step may be performed after moving the magnetic particles M to any of the first plug 10, the second plug 20, and the third plug 30, or to the container 120 by applying magnetic force. In this way, the fourth plug 40 can discharge from the tube 200 without the magnetic particles M being contained in the elution liquid. The fourth plug 40 can discharge from the tube 200 more easily after the magnetic particles M are moved to the second plug 20 or the container 120 because in this case the magnetic particles M do not easily enter the oil of the third plug 30 even without the applied magnetic force.

### 4. NUCLEIC ACID EXTRACTION APPARATUS

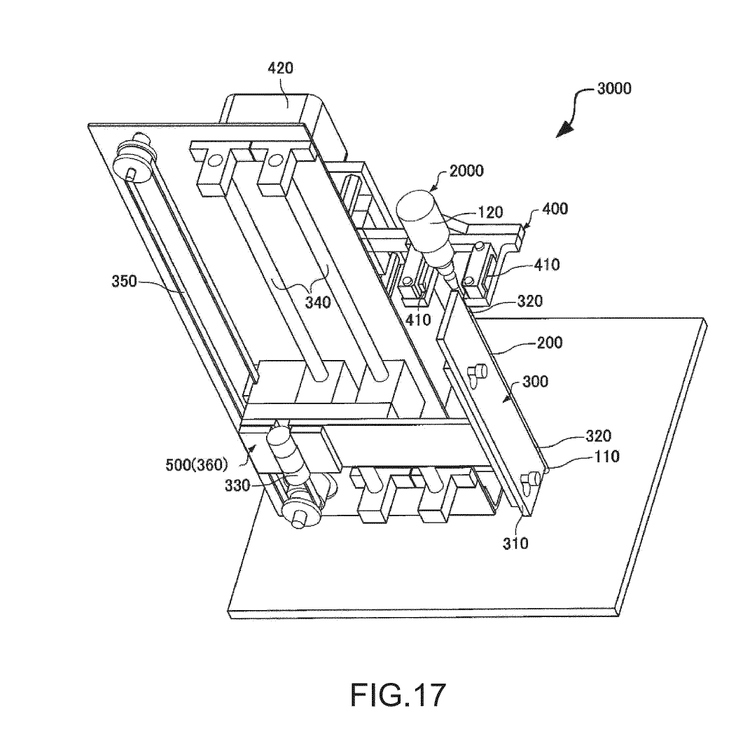

The nucleic acid extraction apparatus according to the present embodiment can preferably be used for the nucleic acid extraction device, the nucleic acid extraction kit, and the nucleic acid extraction method described above. The following embodiment is based on a nucleic acid extraction apparatus 3000 that performs nucleic acid extraction with the nucleic acid extraction kit 2000 installed in the apparatus. FIG. 17 is a perspective view schematically illustrating the nucleic acid extraction apparatus 3000 of the present embodiment.

The nucleic acid extraction apparatus 3000 of the present embodiment includes a mount section 300 that mounts a tube that has a longitudinal direction and in which are disposed, in this order, the first plug 10 formed of oil, the second plug 20 formed of a first washing liquid immiscible with oil, the third plug 30 formed of oil, the fourth plug 40 formed of an elution liquid immiscible with oil, and the fifth plug 50 formed of oil; a magnetic force applying section 400 that applies magnetic force over the side surface of the tube 200 mounted on the mount section 300; and a moving mechanism 500 that moves the relative positions of the mount section 300 and the magnetic force applying section 400 along the longitudinal direction of the tube 200.

The tube 200 mounted on the mount section 300 of the nucleic acid extraction apparatus 3000 is the tube 200 described above. The nucleic acid extraction apparatus 3000 has the mount section 300 on which the tube 200 is mounted. Here, the tube 200 is described as containing the first plug 10 to the fifth plug 50, but may additionally contain the sixth plug 60 and the seventh plug 70.

The mount section 300 is where the tube 200 is mounted. The container 120 joined to the tube 200 may also be mounted on the mount section 300 with the tube 200. The configuration, the mount mechanism, or other structures of the mount section 300 may be designed to such an extent that the magnetic force applying section 400 can apply magnetic force to the tube 200, and, as desired, to the container 120. The mount section 300 may be configured so that the tube 200 can be mounted by being stretched in a straight shape, when the tube 200 is flexible and bent. In the example shown in the figure, the mount section 300 has a brace 310 disposed in a manner that conforms to the shape of the tube 200. The brace 310 is not an essential component; however, installing the brace 310 may help suppress vibrations or the like of the tube 200. In the example shown in the figure, the mount section 300 has a clip mechanism 320, enabling the tube 200 to be fixed at two points.

The mount section 300 is configured so that its position varies relative to the magnetic force applying section 400 along the longitudinal direction of the tube 200. When the mount section 300 is designed to move relative to the magnetic force applying section 400 without moving the magnetic force applying section 400, the mount section 300 is configured to include a moving mechanism 360 (moving mechanism 500) that moves the mount section 300. When the magnetic force applying section 400 has a moving mechanism, it may not be necessary to provide the moving mechanism 360 for the mount section 300. In the example shown in the figure, the mount section 300 is configured to include a hinge 330, guide rails 340, a driving belt 350, and a motor (not illustrated).

The nucleic acid extraction apparatus 3000 of this exemplary embodiment includes only one mount section 300, but may include more than one mount section 300. In this case, more than one magnetic force applying section 400 may be provided, and the plurality of mount sections 300 may be independently provided, or may synchronize.

The magnetic force applying section 400 is configured to apply magnetic force to the tube 200, and, as desired, to the container 120 upon the tube 200 being mounted on the mount section 300. The magnetic force applying section 400 is configured to include, for example, a permanent magnet, an electromagnet, or a combination of these. The magnetic force applying section 400 includes at least one magnet or the like, and more than one magnet or the like may be provided. Preferably, the magnetic force applying section 400 uses a permanent magnet, not an electromagnet, because permanent magnets involve less heat. The permanent magnet may be, for example, nickel-, iron-, cobalt-, samarium-, or neodymium-based magnets.

The magnetic force applying section 400 serves to apply magnetic force to the magnetic particles M present in the container 120 and the tube 200. The magnetic particles M can move inside the container 120 and the tube 200 as the relative positions of the mount section 300 and the magnetic force applying section 400 vary.

In the example shown in the figure, the magnetic force applying section 400 has a pair of permanent magnets 410 disposed opposite each other on the both sides of the container 120 and the tube 200. The permanent magnets 410 are separated from each other by a distance larger than the outer diameter of the tube 200. The direction of the polarity of the permanent magnets 410 is not particularly limited. The magnetic force applying section 400 is configured so that its position varies relative to the mount section 300 along the longitudinal direction of the tube 200. When the magnetic force applying section 400 is designed to move relative to the mount section 300 without moving the mount section 300, the magnetic force applying section 400 is configured to include a moving mechanism (moving mechanism 500) that moves the magnetic force applying section 400.

In the example shown in the figure, the magnetic force applying section 400 is disposed so that one of the permanent magnets 410 moves away from the tube 200 as the other permanent magnet 410 approaches the tube 200. A motor 420 can vibrate the permanent magnets 410 toward and away from the tube 200. By driving the motor 420, the magnetic particles M can be moved back and forth in the tube 200 in directions orthogonal to the longitudinal direction of the tube 200.

The motor 420 may also be driven when applying magnetic force to any positions of the container 120 and the tube 200, as desired. However, the efficiency of washing the magnetic particles M, and the elution efficiency in the tube 200 can be improved by driving the motor 420 when the permanent magnets 410 are at the second plug 20 and the fourth plug 40 of the tube 200.

The nucleic acid extraction apparatus 3000 of the present embodiment enables automating the PCR pretreatment, and can greatly reduce the time and labor required for the pretreatment. The nucleic acid extraction apparatus 3000 of the present embodiment also enables swinging the magnetic force applying section 400, and can wash (purify) the nucleic acid-adsorbing magnetic particles M with improved efficiently for improved PCR accuracy.

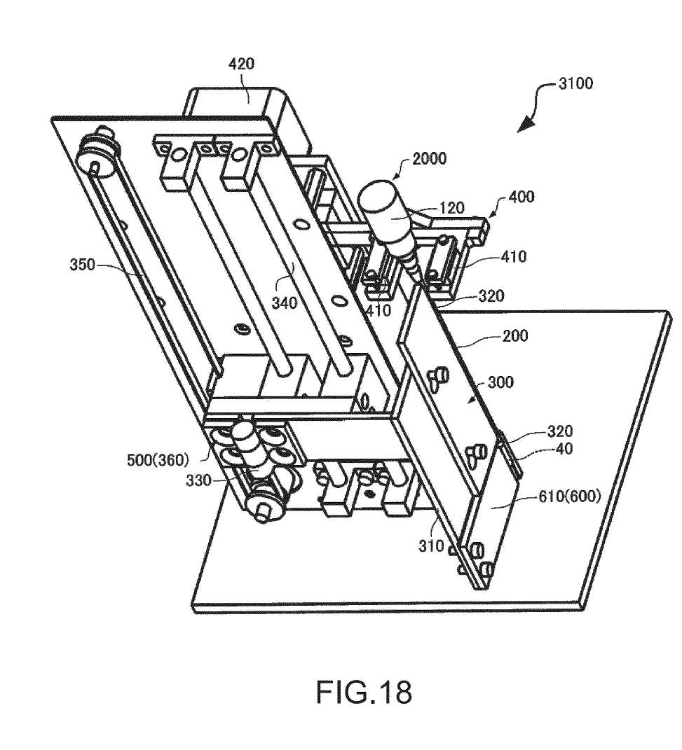

FIG. 18 is a perspective view schematically illustrating a nucleic acid extraction apparatus 3100 according to a variation of the nucleic acid extraction apparatus. The nucleic acid extraction apparatus 3100 is no different from the nucleic acid extraction apparatus 3000 except for the heating section 600, and the functionally same members are referred to by the same reference numerals, and will not be described.

The heating section 600 is configured to heat a part of the tube 200 upon the tube 200 being mounted on the mount section 300. The heating section 600 may be, for example, a heat source, a heat block, a heater, and a coil for electromagnetic heating. The heating section 600 may have any shape, as long as the liquid inside the tube 200 can be heated. For example, the heating section 600 may have a shape that allows for insertion of the tube 200, or a shape in contact with the side surface of the tube 200.

The portions of the tube 200 heated by the heating section 600 include the portion where the fourth plug 40 exists along the longitudinal direction of the tube 200. The heating section 600 may heat other portions of the tube 200. However, the heating section 600 preferably does not heat the portion where the second plug 20 exists along the longitudinal direction of the tube 200.

In the nucleic acid extraction apparatus 3100 illustrated in FIG. 18, a heater 610 juxtaposed with the brace 310, and that heats positions including the fourth plug 40 of the tube 200 is provided as the heating section 600. The heater 610 is shaped so that it contacts about a half of the outer circumference of the tube 200.

The nucleic acid extraction apparatus 3100 enables eluting sufficient amounts of nucleic acids into the elution liquid of the fourth plug 40 even when the amount of the nucleic acids adsorbed on the magnetic particles M has reduced after the washing by at least one of the first washing liquid of the second plug 20 and the second washing liquid of the sixth plug 60. This makes it possible to improve the washing effect, and to elute sufficient concentrations of nucleic acids into the elution liquid for PCR.

### 5. EXPERIMENT EXAMPLES

Embodiments of the invention are described below in greater detail using Experiment Examples. The invention, however, is in no way limited by the following Experiment Examples.

### 5.1. Experiment Example 1

Experiment Example 1 used the nucleic acid extraction kit 2000 of the configuration with the first plug 10 to the seventh plug 70 contained in the tube 200.

First, an adsorption liquid (375 μL), and a magnetic beads dispersion (1 μL) were contained in a 3-mL polyethylene container. The composition of the adsorption liquid was an aqueous solution of 76 mass % guanidine hydrochloride, 1.7 mass % EDTA.2Na dihydrate, and 10 mass % polyoxyethylene sorbitan monolaurate (Toyobo product MagExtractor -Genome-, NPK-1). The magnetic beads dispersion contained 50 volume % of magnetic silica particles, and 20 mass % of lithium chloride.

Blood collected from human (50 μL) was placed in the container through the container opening with a pipette, and the container was stirred by shaking it by hand for 30 seconds after installing a lid. The lid was removed, and the container was joined to the tube. The tube had stoppers at the both ends, and the container was joined to the tube after removing the stopper closing the first plug side of the tube.

The first, third, seventh, and fifth plugs were silicon oils. The first washing liquid of the second plug was an aqueous solution of 76 mass % guanidine hydrochloride. The second washing liquid of the sixth plug was a tris-HCl buffer (solute concentration 5 mM) of pH 8.0. The elution liquid of the fourth plug was sterile water.

The magnetic beads in the container were introduced into the tube by moving a permanent magnet with a hand. The magnetic beads were moved to the fourth plug. The retention time of the magnetic beads in each plug of the tube was 3 seconds in the first, third, and seventh plugs, 20 seconds in the second plug, 20 seconds in the sixth plug, and 30 seconds in the fourth plug. The additional procedures, including vibrating the magnetic beads, were not performed in the second and sixth plugs. The second plug, the sixth plug, and the fourth plug had 25 μL, 25 μL, and 1 μL volumes, respectively.

Thereafter, the stopper on the fifth plug side of the tube was removed, and the container was deformed by hand to discharge the fifth plug and the fourth plug into a PCR reaction container. This procedure was performed after moving and evacuating the magnetic beads to the second plug with the permanent magnet.

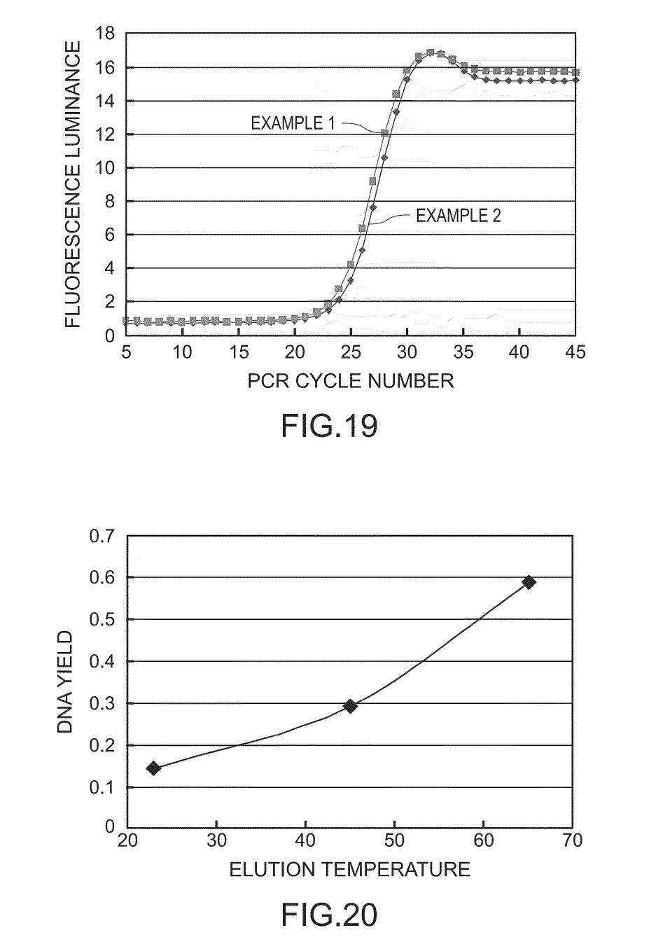

A real-time PCR was then performed by using an ordinary method after adding PCR reaction reagents (19 μL) to the extract. The PCR reaction reagents contained 4 μL of LightCycler 480 Genotyping Master (Roche Diagnostics product 4 707 524), 0.4 μL of SYBR Green I (Life Technologies product S7563) diluted 1000 times with sterile water, 100 μM β actin detection primers (F/R; 0.06 μL each), and 14.48 μL of sterile water. The PCR amplification curve of Experiment Example 1 is shown in FIG. 19. In FIG. 19, the vertical axis represents fluorescence luminance, and the horizontal axis represents PCR cycle number.

### 5.2. Experiment Example 2

In Experiment Example 2, nucleic acid extraction was performed by using a common nucleic acid extraction technique.

First, an adsorption liquid (375 μL), and a magnetic beads dispersion (20 μL) were contained in a 1.5-mL polyethylene container. The adsorption liquid and the magnetic beads dispersion had the same compositions as in Experiment Example 1.

Thereafter, blood collected from human (50 μL) was introduced into the container through the container opening with a pipette. After installing a lid, the container was stirred with a vortex mixer for 10 minutes, and subjected to B/F separation with a magnetic stand and a pipette. The magnetic beads and a small amount of adsorption liquid remained in the container after this procedure.

A first washing liquid (450 μL) of the same composition used in Experiment Example 1 was introduced into the container, and stirred for 5 seconds with a vortex mixer after installing a lid. The first washing liquid was then removed with a magnetic stand and a pipette. This procedure was repeated twice. The magnetic beads and a small amount of first washing liquid remained in the container after this procedure.

Thereafter, a second washing liquid (450 μL) of the same composition used in Experiment Example 1 was introduced into the container, and stirred for 5 seconds with a vortex mixer after installing a lid. The second washing liquid was then removed with a magnetic stand and a pipette. This procedure was repeated twice. The magnetic beads and a small amount of second washing liquid remained in the container after this procedure.

After adding sterile water (elution liquid) 50 μL to the container, the container was closed with a lid, and stirred for 10 minutes with a vortex mixer. The supernatant liquid was then collected with a magnetic stand and a pipette. The supernatant liquid contained the target nucleic acid.

A 1-μL aliquot of the extract was dispensed, and a real-time PCR was performed by using an ordinary method after adding 19 μL of PCR reaction reagents. The PCR reaction reagents contained 4 μL of LightCycler 480 Genotyping Master (Roche Diagnostics product 4 707 524), 0.4 μL of SYBR Green I (Life Technologies product S7563) diluted 1000 times with sterile water, 100 μm β actin detection primers (F/R; 0.06 μL each, and 14.48 μL of sterile water. The amplification curve is shown in FIG. 19.

### 5.3. Experiment Example 3

Experiment Example 3 used the nucleic acid extraction kit 2000 of the configuration with the first plug 10 to the fifth plug 50 contained in the tube 200.

The adsorption liquid and the magnetic beads dispersion had the same compositions as in Experiment Example 1, and silicon oils were used for the first, third, and fifth plugs as in Experiment Example 1.

The first washing liquid of the second plug was tris-HCl buffer (solute concentration 5 mM) of pH 8.0. The elution liquid of the fourth plug was sterile water.

Blood collected from human (50 μL) was placed in the container through the container opening with a pipette, and the container was stirred by shaking it for 30 seconds by hand after installing a lid. The lid was removed, and the container was joined to the tube. The tube had stoppers at the both ends, and the container was joined to the tube after removing the stopper closing the first plug side of the tube.

The magnetic beads in the container were introduced into the tube by moving a permanent magnet by hand. The magnetic beads were moved to the fourth plug. The retention time of the magnetic beads in each plug of the tube was 3 seconds in the first and third plugs, 20 seconds in the second plug, and 30 seconds in the fourth plug. The additional procedures, including vibrating the magnetic beads, were not performed in the second plug. The second plug and the fourth plug had 25 μL and 1 μL volumes, respectively.

Thereafter, the stopper on the fifth plug side of the tube was removed, and the container was deformed by hand to discharge the fifth plug and the fourth plug into a PCR reaction container. This procedure was performed after moving and evacuating the magnetic beads to the second plug with the permanent magnet.

A real-time PCR was then performed by using an ordinary method after adding PCR reaction reagents (19 μL) to the extract. The PCR reaction reagents contained 4 μL of LightCycler 480 Genotyping Master (Roche Diagnostics product 4 707 524), 0.4 μL of SYBR Green I (Life Technologies product S7563) diluted 1000 times with sterile water, 100 μM β actin detection primers (F/R; 0.06 μL each), and 14.48 μL of sterile water.

The amplification curve had nearly the same characteristics as that shown in FIG. 19. When the experiment of this Experiment Example was conducted by using 76 mass % guanidine hydrochloride for the first washing liquid of the second plug, the threshold occurred at least 10 cycles later than in the amplification curve of Experiment Example 1.

### 5.4. Experiment Example 4

### Effect of Elution Temperature on DNA Yield

Experiment Example 4 performed nucleic acid extraction by using a common nucleic acid extraction technique.

First, an adsorption liquid (375 μL), and a magnetic beads dispersion (20 μL) were contained in a 1.5-mL polyethylene container. The adsorption liquid and the magnetic beads dispersion had the same compositions as in Experiment Example 1.

Thereafter, a genomic DNA solution (50 μL) concentrated to 1 ng/μL was introduced into the container through the container opening with a pipette. After installing a lid, the container was stirred with a vortex mixer for 10 minutes, and subjected to B/F separation with a magnetic stand and a pipette. The magnetic beads and a small amount of adsorption liquid remained in the container after this procedure.

A first washing liquid (450 μL) of the same composition used in Experiment Example 1 was introduced into the container, and stirred for 5 seconds with a vortex mixer after installing a lid. The first washing liquid was then removed with a magnetic stand and a pipette. This procedure was repeated twice. The magnetic beads and a small amount of first washing liquid remained in the container after this procedure.

Thereafter, a second washing liquid (450 μL) of the same composition used in Experiment Example 1 was introduced into the container, and stirred for 5 seconds with a vortex mixer after installing a lid. The second washing liquid was then removed with a magnetic stand and a pipette. This procedure was repeated twice. The magnetic beads and a small amount of second washing liquid remained in the container after this procedure.

After adding sterile water (elution liquid) 50 to the container, the container was closed with a lid, and stirred for 5 seconds with a vortex mixer. The container was then heated for 2 minutes with a tube heater, and stirred again for 10 seconds with a vortex mixer. The supernatant liquid was then collected with a magnetic stand and a pipette. Here, the heating by the tube heater was performed at three different temperatures, 23° C. (allowed to stand at room temperature), 45° C., and 65° C.

A 1-μL aliquot of the extract was dispensed, and a real-time PCR was performed by using an ordinary method after adding 19 μL of PCR reaction reagents. A genomic DNA solution concentrated to 1 ng/μL was also used as a PCR reaction sample for comparison. The PCR reaction reagents contained 4 μL of LightCycler 480 Genotyping Master (Roche Diagnostics product 4 707 524), 0.4 μL of SYBR Green I (Life Technologies product S7563) diluted 1000 times with sterile water, 100 μM β actin detection primers (F/R; 0.06 μL each), and 14.48 μL of sterile water.

FIG. 20 represents the relationship between elution temperature and DNA yield. The result was obtained after calculations from the threshold cycles of real-time PCR. DNA yield is represented by the expression 2^{(Ct0 Ct1)} as a ratio with respect to the comparative sample (taken as 1), where Ct0 is the threshold cycle of the comparative sample, and Ct1 is the threshold cycle of the extraction sample.

### 5.5. Experiment Example 5

Example 5 examined how the distance from the plugs in the nucleic acid extraction device to a hand wearing a nitrile glove (charged substance) affects the displacement or disruption of the plugs inside the tube.

First, ten polypropylene tubes measuring 1 mm in inner diameter and 3 mm in outer diameter were prepared. Each tube was charged with a silicone oil having a kinetic viscosity of 2 cSt (25° C.), and 1 μL of purified water. The tube was then grabbed with a hand wearing a nitrile glove in a portion charged with the silicone oil and water, and stroked 10 times with the gloved hand. The number of tubes was then counted in which a 1 mm or more displacement occurred in the water liquid level, and in which the water disrupted. The all ten tubes had a liquid level displacement or plug disruption.

Thereafter, ten polypropylene tubes measuring 3 mm in inner diameter and 5 mm in outer diameter, and ten polypropylene tubes measuring 1 mm in inner diameter and 3 mm in outer diameter were prepared. The polypropylene tubes with 1-mm inner diameter and 3-mm outer diameter were each inserted into the inner cavity of each polypropylene tube of 3-mm inner diameter and 5-mm outer diameter to obtain ten tubes measuring 1 mm in inner diameter and 5 mm in outer diameter.

Each tube was charged with a silicone oil with a kinetic viscosity of 2 cSt (25° C.) and 1 μL of purified water. The tube was then grabbed with a hand wearing a nitrile glove in a portion charged with the silicone oil and water, and stroked 10 times with the gloved hand. The number of tubes was then counted in which a 1 mm or more displacement occurred in the water liquid level, and in which the water disrupted. Nine out of the ten tubes had a liquid level displacement or plug disruption.

Thereafter, ten polypropylene tubes measuring 5 mm in inner diameter and 7 mm in outer diameter, ten polypropylene tubes measuring 3 mm in inner diameter and 5 mm in outer diameter, and ten polypropylene tubes measuring 1 mm in inner diameter and 3 mm in outer diameter were prepared. The polypropylene tubes with 3-mm inner diameter and 5-mm outer diameter were each inserted into the inner cavity of each polypropylene tube of 5-mm inner diameter and 7-mm outer diameter, and The polypropylene tubes with 1-mm inner diameter and 3-mm outer diameter were each inserted into the inner cavity of each polypropylene tube of 3-mm inner diameter and 5-mm outer diameter to obtain ten tubes measuring 1 mm in inner diameter and 7 mm in outer diameter.

Each tube was charged with a silicone oil with a kinetic viscosity of 2 cSt (25° C.) and 1 μL of purified water. The tube was then grabbed with a hand wearing a nitrile glove in a portion charged with the silicone oil and water, and stroked 10 times with the gloved hand. The number of tubes was then counted in which a 1 mm or more displacement occurred in the water liquid level, and in which the water disrupted. None of the ten tubes had a liquid level displacement or plug disruption.

### 5.6. Experiment Results

The Experiment Examples revealed the following.

(1) By comparing the time needed for the nucleic acid extraction performed as a PCR pretreatment, the time from the insertion of the specimen into the container to the introduction of the target nucleic acid into the PCR reaction container was about 2 minutes in Experiment Example 1, whereas it took about 30 minutes in Experiment Example 2. It was found from this result that the nucleic acid extraction method of Experiment Example 1 required a much shorter nucleic acid extraction time than the nucleic acid extraction method of Experiment Example 2.

(2) Each washing liquid used in Experiment Example 1 was about 1/18 of the volume used in Experiment Example 2. The elution liquid used in Experiment Example 1 was about 1/50 of the volume used in Experiment Example 2. That is, it was found that the volumes of the washing liquid and the elution liquid used in Experiment Example 1 were sufficient despite the much smaller volumes than those used in Experiment Example 2.

(3) By comparing the target nucleic acid concentration in the elution liquid in terms of the volumes of the adsorption liquid and the elution liquid, the concentration should ideally be 50 times higher in Experiment Example 1 than in Experiment Example 2. However, such a 50-fold concentration difference was not observed between Experiment Example 1 and Experiment Example 2 in the foregoing Experiment Examples because the amount of nucleic acids contained in the blood sample exceeded the amount that can be adsorbed by 1 μL of magnetic beads, and the nucleic acids contained in the blood sample could not be fully collected. The concentration will be 50 times higher in Experiment Example 1 than in Experiment Example 2 when the amount of the nucleic acids contained in the specimen is small, and does not exceed the amount that can be adsorbed by 1 μL of magnetic beads.

(4) From the graph of FIG. 19, it was found that the threshold of the nucleic acid amplification rate occurred about 0.6 cycles earlier in Experiment Example 1 than in Experiment Example 2 with the whole blood sample that also had a large nucleic acid content. Specifically, the target nucleic acid concentration was higher in the PCR reaction liquid of Experiment Example 1 than the PCR reaction liquid used in Experiment Example 2. This is supportive of the target nucleic acid concentration in the elution liquid being higher in Experiment Example 1 than in Experiment Example 2.

(5) It was found from the result of Experiment Example 3 that a sufficient extraction was also possible when a buffer was used for the second plug. It was also found that the threshold of the PCR amplification curve occurred notably late when the second plug was a guanidine aqueous solution and inhibited the enzyme reaction. Another finding is that diluting the extract at least 1000 times can reduce the enzyme reaction inhibition by the guanidine aqueous solution.

(6) It was found from the result of Experiment Example 4 that increasing the fourth plug to about 40° C. and higher produced DNA in good yield, sufficient for use in PCR.

(7) It was found from the result of Experiment Example 5 that the liquid level displacement or plug disruption could be suppressed when the charged substance was separated from the reagent-charged liquid portion, specifically the plug portion, by a distance of 3 mm or more.

The invention is not limited to the foregoing exemplary embodiments, and may be modified in many ways. For example, the invention encompasses configurations substantially the same as the configurations described in the embodiments (for example, configurations sharing the same functions, methods, and results, or configurations sharing the same objects and effects). The invention also encompasses configurations that differ from the configurations of the foregoing embodiments in non-substantive parts. The invention also encompasses configurations having the same advantages as the configurations of the foregoing embodiments, or configurations that can achieve the same object as the configurations of the foregoing embodiments. The invention also encompasses configurations that use known techniques with the configurations of the foregoing embodiments.

The entire disclosures of Japanese Patent Application Nos. 2012-086516 filed Apr. 5, 2012 and 2013-239671 filed Nov. 20, 2013 are expressly incorporated by reference herein.

What is claimed is:

1. A nucleic acid extraction device comprising:
a tube section, the tube section having disposed therein, in this order:
a first plug formed of oil;
a second plug formed of a washing liquid immiscible with oil and suitable for washing a substance adsorbing a nucleic acid;
a third plug formed of oil;
a fourth plug formed of an elution liquid immiscible with oil and suitable for eluting the nucleic acid from the substance; and
a fifth plug formed of oil; and
a cover disposed around the tube section.

2. The nucleic acid extraction device according to claim 1, wherein the cover is detachable from the tube section.

3. The nucleic acid extraction device according to claim 1, wherein the cover is expandable and compressible in an extending direction of the tube section.

4. The nucleic acid extraction device according to claim 1, wherein the tube section and the cover are separated from each other.

5. The nucleic acid extraction device according to claim 1, wherein a distance from an inner cavity surface of the tube section to an outer surface of the cover is 3 mm or more.

6. The nucleic acid extraction device according to claim 1, wherein the cover has a slit that extends along an extending direction of the tube section.

7. The nucleic acid extraction device according to claim 1, wherein the cover has a hole.

8. The nucleic acid extraction device according to claim 1, wherein the cover is made of a deformable material and a gas is sealed between the tube section and the cover to prevent the tube section and the cover from adhering to each other.

9. The nucleic acid extraction device according to claim 1, wherein the cover contains a non-magnetic substance selected from metals and metal alloys.

10. The nucleic acid extraction device according to claim 1, wherein a side wall of the tube section contains a non-magnetic substance selected from metals and metal alloys.

11. A nucleic acid extraction device comprising:
a tube section, the tube section having disposed therein:
a first plug formed of oil; and
a second plug formed of an aqueous liquid immiscible with the oil; and
a cover disposed around the tube section.

12. The nucleic acid extraction device according to claim 11, wherein the cover is detachable from the tube section.

13. The nucleic acid extraction device according to claim 11, wherein the cover is expandable and compressible in an extending direction of the tube section.

14. The nucleic acid extraction device according to claim 11, wherein the tube section and the cover are separated from each other.

15. The nucleic acid extraction device according to claim 11, wherein a distance from an inner cavity surface of the tube section to an outer surface of the cover is 3 mm or more.

16. The nucleic acid extraction device according to claim 11, wherein the cover has a slit that extends along an extending direction of the tube section.

17. The nucleic acid extraction device according to claim 11, wherein the cover has a hole.

18. The nucleic acid extraction device according to claim 11, wherein the cover is made of a deformable material and a gas is sealed between the tube section and the cover to prevent the tube section and the cover from adhering to each other.

19. A nucleic acid extraction device comprising:
a tube section, the tube section having disposed therein, in this order:
a first plug formed of oil;
a second plug formed of a washing liquid immiscible with oil and suitable for washing a substance adsorbing a nucleic acid;
a third plug formed of oil;
a fourth plug formed of an elution liquid immiscible with oil and suitable for eluting the nucleic acid from the substance; and
a fifth plug formed of oil,
the tube section having a side wall with a thickness of 3 mm or more.

20. A nucleic acid extraction device comprising:
a tube section, the tube section having disposed therein:
a first plug formed of oil; and
a second plug formed of an aqueous liquid immiscible with the oil,
the tube section having a side wall with a thickness of 3 mm or more.
